(19) United States
(12) Patent Application Publication
(10) Pub. No.: US 2010/0273876 A1
(43) Pub. Date: Oct. 28, 2010

(54) ANTIBACTERIAL FORMULATION COMPRISING A DIALKYL SULPHOSUCCINATE AND A CARBANILIDE ANTIBACTERIAL AGENT

(76) Inventors: Scott Seville, West Yorkshire (GB); Stephen Philip Jones, Surrey (GB); Daniel James Fitzgerald, Leeds (GB); Nayan N. Desai, Santa Rosa, CA (US); Refika I. Pakunlu, Petaluma, CA (US)

Correspondence Address: Weaver Austin Villeneuve & Sampson LLP, P.O. BOX 70250, OAKLAND, CA, 94612-0250, US

(21) Appl. No.: 12/600,733

(22) PCT Filed: Jun. 2, 2008
(86) PCT No.: PCT/GB2008/001896

§ 371 Date: Jun. 9, 2010

(30) Foreign Application Priority Data
May 31, 2007 (GB) 0710346.8
May 31, 2007 (GB) 0710350.0

(51) Int. Cl. A61K 31/225; A01N 37/36; A01P 1/00; A61P 31/04; A61P 17/10

(52) U.S. Cl. 514/547

(57) ABSTRACT

Antibacterial formulation containing (a) a dialkyl sulphosuccinate (DAS) or derivative thereof and (b) a carbanilide antibacterial agent such as triclocarban (TCC). The formulation may be for use in the treatment of a bacterial condition, in particular acne, body odour or a condition affecting the oral cavity. It may contain, in addition to the DAS and the carbanilide, (i) a poly-oxyalkylene-based solubilising agent; (ii) an organic solvent; (iii) a thickening agent; and (iv) water.

### FIELD OF THE INVENTION

This invention relates to antibacterial formulations, and to the use of certain combinations of compounds as antibacterial agents.

### BACKGROUND TO THE INVENTION

Dialkyl sulphosuccinates (DASs) are known anionic surfactants. In particular dioctyl sulphosuccinate (DOS) and its salts, more particularly sodium DOS, are used as wetting, solubilising, dispersing and emulsifying agents, for example in household cleaning products. The sodium salt is also known for use as a mild contact laxative. Synonyms for DOS include bis(2-ethylhexyl)sulphonate; 2-sulpho-succinic acid dioctyl ester; 1,4-bis(n-octyl)sulphobutanedioate and 1,4-bis(octyloxy)-1,4-dioxo-2-butanesulphonic acid, and its salts are commercially available under a range of trade names such as Docusate and Duosol.

Dioctyl sodium sulphosuccinate is also known for use as a stabiliser and/or surfactant in formulations containing other active substances, including in anti-acne formulations containing peroxides such as benzoyl peroxide and/or antibiotics such as erythromycin—see GB-2 054 375, U.S. Pat. No. 5,767,098, U.S. Pat. No. 4,497,794 and US-2003/0044432—and in anti-ageing formulations containing retinoids.

In WO-86/07258, DASs, in particular dioctyl sodium sulphosuccinate, are also proposed for use in the treatment of bacterial infections of humans and animals. The document describes topical formulations for use as skin cleansers and as lotions for the treatment of acne. The dioctyl sodium sulphosuccinate is used however at relatively low concentrations, in the examples at 0.2% w/w. Potentially undesirable side effects are cited as a disincentive to using higher concentrations.

Moreover dioctyl sodium sulphosuccinate is also poorly water soluble, which can limit its concentration in aqueous-based formulations. As a surfactant, the substance is typically used at a concentration of 1% w/w or lower. In skin formulations it is regarded by the FDA as an inactive ingredient when used at concentrations of 2 or 3% w/w.

Triclocarban, also called 3,4,4′-trichlorocarbanilide; 3,4,4′-trichlorodiphenyl urea; TCC and N-(4-chlorophenyl)-N′-(3,4-dichlorophenyl) urea, amongst other synonyms, is a known disinfectant and antibacterial agent. It is used, for example, for cleansing and disinfecting skin, in particular in hospitals, and also in cosmetics and personal care products, in household goods and in some plastics and textile goods. U.S. Pat. No. 6,224,886 also discloses its use in a topical anti-acne formulation, although in the absence of anionic surfactants and with a reference to the antibacterial activity of the TCC being improved when detergent levels are reduced.

WO-02/02073 further discloses anti-sebum skin care compositions containing TCC, which must contain not more than 5% of a surfactant and not more than 20% of an alcohol, the TCC itself being present at from about 0.001 to about 10% but preferably below 2%. This document teaches that alcohols should be avoided in the TCC formulation if possible, as should anionic and nonionic surfactants, the DASs being specifically listed as substances which should be excluded.

In U.S. Pat. No. 7,201,914, TCC is also listed as a potential antimicrobial agent for use in a topical anti-perspirant-containing composition which can be used, inter alia, to treat facial acne.

It is thus known to combine TCC with surfactants, including certain types of anionic surfactant, in topical skin treatment formulations. U.S. Pat. No. 6,121,214 describes the use of TCC as an antibacterial agent in a liquid soap, in combination with a polyethylene glycol (PEG) glyceride solubiliser and optionally also disodium laureth sulphosuccinate. This document does not however refer to any other types of sulphosuccinate surfactant.

U.S. Pat. No. 5,883,059 discloses topical antibacterial cleansing compositions which contain an antibacterial agent such as TCC, an anionic surfactant and an amphoteric surfactant. The anionic surfactant can be DOS; it is preferably present at between 10 and 12% w/w.

Other halogenated carbanilides are also known for use as antimicrobial agents. Examples include 3-trifluoromethyl-4,4′-dichlorocarbanilide and 3,3′,4-trichlorocarbanilide.

U.S. Pat. No. 5,977,049 for example describes the use of a carbanilide antibacterial agent, in particular TCC, in an aqueous cleansing composition. The carbanilide is said to be solubilised and stabilised by using a primary ethoxylated alcohol. The composition also contains an anionic surfactant in a cleansing effective quantity; sulphosuccinates are not listed as suitable surfactants for use in this context.

U.S. Pat. No. 5,922,768 discloses a personal cleansing composition containing an antibacterial aromatic carbanilide, again preferably TCC, together with a water soluble polyethylene glycol and a fragrance. Anionic surfactants, including alkyl sulphosuccinates, are mentioned as potential additives.

TCC, like DASs, is also poorly soluble in water and many water/organic solvent mixtures. It can therefore be difficult to formulate at effective concentrations in aqueous-based formulations.

It has now surprisingly been found that when a DAS is combined with a carbanilide antibacterial agent such as TCC, a potentiating effect can be observed on their combined level of antibacterial activity, in particular versus propionibacteria and more particularly versus Propionibacterium acnes, the bacterium implicated in inflammatory acne. This is particularly surprising in view of the teachings of U.S. Pat. No. 6,224,886 and WO-02/02073 discussed above. As a result, novel antibacterial and/or anti-acne formulations can be prepared, in particular for topical application, either with improved efficacy and/or containing lower levels of at least one of the active ingredients than would previously have been thought necessary.

Moreover, a novel formulation has been developed in which both a DAS and a carbanilide antibacterial agent can be solubilised and stabilised at concentrations suitable for topical antibacterial use, so as to make effective use of the antibacterial synergy observed for this new combination of actives.

### STATEMENTS OF THE INVENTION

According to a first aspect of the present invention there is provided an antibacterial formulation containing (a) a dialkyl sulphosuccinate (DAS) or derivative thereof and (b) a carbanilide antibacterial agent.

In an embodiment, the formulation is free from amphoteric surfactants.

This formulation is preferably suitable for topical application to, and/or contact with, the skin, in particular human skin. The DAS or derivative and the carbanilide antibacterial agent are therefore preferably contained in a pharmaceutically acceptable vehicle which can safely be applied to, and/or contacted with, the skin. Ideally the formulation is suitable for topical application to areas such as the nares, eyes, scalp and/or vagina, and/or to tissue surfaces within the ears. It may be suitable for topical application to one or more tissue surfaces within the mouth. It may in particular be suitable for topical application to such surfaces in or on the human body.

A formulation which is “suitable for” topical application may also be adapted for topical application.

A formulation which is applied topically within the oral cavity will not, in the course of its ordinary usage, be intentionally swallowed for the purpose of systemically administering a substance contained within it, but rather, will be retained in the oral cavity, in contact with the relevant oral tissue and/or dental surface, for sufficient time as to exert a pharmaceutical effect, for example an antibacterial and/or anti-plaque effect.

Suitable vehicles for topical formulations will be well known to those skilled in the art of preparing topical skin care or pharmaceutical preparations. The vehicle will typically be a fluid, which term includes a cream, paste, gel, lotion, foam, ointment, varnish or other viscous or semi-viscous fluid, as well as less viscous fluids such as might be used in sprays (for example for nasal or oral use), drops (for example for use in the eyes or ears), aerosols or mouthwashes. The DAS or derivative and the carbanilide antibacterial agent may each independently be present in the form of a solution or suspension, the term “suspension” including emulsions, micellar systems and other multi-phase dispersions.

Either or both of the DAS or derivative and the carbanilide antibacterial agent may, whether separately or together, be carried in or on a delivery vehicle which is suitable for targeting or controlling its release at the intended site of administration. Such vehicles include liposomes and other encapsulating entities, for example niosomes, aspasomes, microsponges, microemulsions, hydrogels and solid lipid nanoparticles.

The term “dialkyl sulphosuccinate” or “DAS” as used herein means a dialkyl ester of sulphosuccinic acid, in which the sulphonic acid group is present as —S(O)₂—OH. Derivatives of dialkyl sulphosuccinates can include salts such as metal salts, ammonium salts (in particular the NH₄⁺ salt) and any form of the compound in which the sulphonic acid group is present as the sulphonate —S(O)₂—O⁻, as well as solvates and so-called “prodrug” forms which revert to an active form of the compound at an appropriate time on or after administration.

The two alkyl groups of the DAS may be for example C₁ to C₁₈ alkyl groups, in particular C₂ to C₁₂ or C₂ to C₁₀ alkyl groups, such as C₆ to C₁₀ and in particular C₈. Thus the DAS is suitably a dioctyl sulphosuccinate as described above. The alkyl groups may be substituted or unsubstituted, preferably the latter. If substituted, they may for example be mono-, di- or tri-substituted, and may suitably include one or more substituents selected from amido groups and ethers. They may be either straight chain or branched. They may include one or more unsaturated carbon-carbon bonds. In an embodiment of the invention, the alkyl groups of the DAS are not substituted with ether groups.

The DAS or derivative is conveniently used in the form of a salt in which the sulphonic acid group is present as the sulphonate —S(O)₂—O⁻, such as in particular a metal salt or ammonium salt. Suitable metal salts include the alkali metal salts (for example the sodium or potassium salts, in particular the former) and the alkaline earth metal salts (for example the calcium salt).

The DAS or derivative is suitably pharmaceutically acceptable (which term includes suitable for veterinary use).

In an embodiment of the invention, the DAS is used in the form of its sodium salt, for example dioctyl sodium sulphosuccinate (also known as dioctyl sulphosuccinate sodium or docusate sodium).

The term “carbanilide antibacterial agent” as used herein means a carbanilide or derivative thereof which is active as an antibacterial agent, against at least one bacterium or bacterial strain.

Carbanilide itself is also known as 1,3-diphenylcarbamide; N,N′-diphenylurea; and acardite. For use in the present invention, a carbanilide is suitably a halogenated carbanilide, in which either or both (preferably both) of the phenyl groups are independently substituted with one or more halogens. It may in particular be a chlorinated carbanilide, in which either or both (preferably both) of the phenyl groups are independently substituted with one or more chlorine groups.

The carbanilide antibacterial agent may for example be selected :from triclocarban (TCC), 3-trifluoromethyl-4,4′-dichlorocarbanilide and 3,3′,4-trichlorocarbanilide.

More particularly it is TCC, which has the following formula:

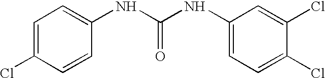

(CAS Registry No. 101-20-2).

A carbanilide or derivative is suitably pharmaceutically acceptable (which includes suitable for veterinary use).

A “derivative” of either a DAS or a carbanilide may be a pharmaceutically acceptable (which term includes acceptable for veterinary use) derivative. It may be for example a salt, complex or solvate or a so-called prodrug form or protected form which reverts to an active form of the relevant compound at an appropriate time on or after administration. In the case of a DAS, a derivative may be a free acid—ie, an alkyl sulphosuccinnic acid—from which the sulphosuccinate can be derived. In an embodiment, however, the DAS is present in the form of the sulphosuccinate. In an embodiment, the carbanilide antibacterial agent is present in the form of a carbanilide alone (ie, of a 1,3-diphenylcarbamide).

A formulation according to the invention may contain a mixture of two or more DASs or derivatives thereof. It may contain a mixture of two or more carbanilide antibacterial agents.

In a formulation according to the invention, the combination of the DAS or derivative and the carbanilide antibacterial agent is present as an active (ie, antibacterially active) agent. The combination may be present as an anti-acne agent (ie, as an agent which is active against acne (which includes against a symptom and/or a cause of acne and/or against one or more micro-organisms associated with acne)). It may be present as an anti-plaque agent, as described in more detail below.

Suitably, the DAS or derivative is not used purely or even primarily as a stabiliser for another substance such as an active ingredient, or as a surface active agent (surfactant), or as a wetting or solubilising or dispersing or emulsifying agent, or as a processing aid.

Surprisingly, a DAS and a carbanilide antibacterial agent such as TCC have been found to act together to inhibit, and often to prevent, bacterial (including propionibacterial) activity. In other words, they have been found to potentiate one another's activity as compared to the sum of the activities of the two agents individually. This is particularly surprising in view of earlier teachings, as discussed above, that an anionic surfactant can reduce the antimicrobial activity of a carbanilide such as TCC.

It is possible that this potentiation of antibacterial activity by a combination of a DAS or DAS derivative and a carbanilide antibacterial agent may be at least partly due to the formation of a reaction product having an antibacterial activity greater than the, sum of those of the individual reactants. The invention may thus embrace an antibacterial formulations containing a reaction product formed between a DAS or derivative thereof and a carbonilide antibacterial agent,in particular between:a DOS such as dioc sodium sulphosuccinate and TCC; this reaction product may be formed in situ immediately prior to, or at the point of, use.

In a formulation according to the invention the DAS or derivative and the carbanilide antibacterial agent, and their relative proportions, are preferably such as to yield at least an additive level of antibacterial activity compared to the activities of the individual compounds alone. An increased level of activity in these contexts may be manifested by a lower concentration of the compound(s) being needed to inhibit and/or to kill the relevant bacterium, and/or by a larger zone of inhibition in a disc diffusion assay (as described below), and/or by a faster rate of bacterial inhibition or killing.

Antibacterial activity encompasses activity against both Gram-positive and Gram-negative bacteria. It may be growth inhibitory activity or more preferably bactericidal (ie, lethal to the relevant organism). It may comprise activity against sessile and/or planktonic bacteria.

In the context of this invention, activity against a particular species of bacterium may be taken to mean activity against at least one, preferably two or more, strains of that species.

Antibacterial activity may be or include the ability to disrupt and/or suppress biofilm formation by the relevant organism.

In the present context, the disruption of biofilm formation embraces any negative effect on the ability of a bacterium to form, maintain or exist in a biofilm, and/or on a biofilm already formed by the bacterium. Thus, it may involve reducing the amount of a previously formed biofilm, and/or impairing such a biofilm. It may involve killing or inhibiting sessile bacteria within a biofilm.

The biofilm may for example be in the form of dental plaque. The disruption may involve altering the bacterial composition of dental plaque to a more healthy state, reducing the amount of plaque by for example inhibiting co-aggregation, and/or interfering with the production of signal molecules that mediate the organisation and integrity of plaque.

Suppression of biofilm formation embraces any degree of impairment (including complete prevention) of the ability of a bacterium to form, or more typically to co-aggregate with, a biofilm. It thus embraces total or partial impairment, including reducing the amount and/or strength of biofilm which the bacterium is able to form and/or the speed with which it is able to do so. It may involve preventing or reducing the growth or the rate of growth of an existing biofilm formed by the bacterium.

An antibacterial formulation according to the present invention is preferably active at least against Gram-positive bacteria, for example against one or more bacteria selected from Propionibacterium spp, staphylococci, bacteria implicated in conditions within the oral cavity and bacteria implicated in body odour. It may in particular be active against one or more of the bacteria referred to below in connection with the fifth to the eighth aspects of the invention.

In an embodiment of the invention, the formulation is active against bacteria associated with skin or skin-borne infections, in particular acne, and/or against bacteria associated with infections within the oral cavity. It may be active against staphylococci (and in cases other Gram-positive cocci such as enterococci), for example Staphylococcus aureus, and/or against propionibacteria, and/or against bacteria associated with dental or periodontal conditions, for example Porphyromonas gingivalis and/or S. mutans.

In a particularly preferred embodiment, it is active against one or more bacteria associated with acne, in particular propionibacteria such as P. acnes and in some instances P. granulosum. In another preferred embodiment, the formulation is active against one or more bacteria associated with conditions affecting the oral cavity, for example P. gingivalis. In another preferred embodiment, the formulation is active against one or more staphylococci, in particular S. aureus. In another preferred embodiment, the formulation is active against one or more bacteria associated with body odour, in particular in the axilla or feet.

The formulation is preferably active against bacteria, in particular propionibacteria, which are wholly or partially resitant to one or more antibiotics, for instance those which are in common clinical use. It is ideally active against macrolide-lincosamide-streptogramin (MLS) resistant and/or macrolide-lincosamide-streptogramin-ketolide (MLSK) resistant strains of bacteria. In particular it may be active against erythromycin-resistant, clindamycin-resistant and/or tetracycline-resistant strains of bacteria, for example P. acnes strains, the term tetracycline here referring to the class of antibiotics including for example minocycline and doxycycline as well as the specific antibiotic known as tetracycline.

In order to extend the activity, for instance against bacteria of low susceptibility, of a formulation according to the invention, it may contain, or be used in combination with, an additional antimicrobial agent, in particular an agent which is active against Gram-negative bacteria. Its activity spectrum may be further extended by the inclusion of, or its use in combination with, one or more antimicrobial agents which are active against pathogenic microfungi. An additional antimicrobial agent may be for either topical or systemic administration, suitably topical.

Antibacterial activity may be measured in conventional manner, for instance using the tests described in the examples below. Generally tests for activity involve treating a culture of the relevant bacterium with the candidate antibacterial compound, incubating the treated culture under conditions which would ordinarily support growth of the bacterium, and assessing the level of growth, if any, which can occur in the presence of the candidate compound.

Preferably the DAS or derivative used in the present invention has a minimum inhibitory concentration (MIC), at least against propionibacteria, of 125 μg/ml or less, more preferably 62.5 or 31.25 or in cases 20 μg/ml or less, such as from 31.25 to 3.9 μg/ml. Its corresponding minimum bactericidal concentration (MBC) is preferably 125 μg/ml or less, preferably 70 μg/ml or less. More preferably the DAS or derivative also exhibits such characteristics in the presence of at least one of, preferably both of, lipid and sodium chloride, for instance as tested in the examples below—these are species which can be present at the surface of the skin and hence performance in this context can be indicative of suitability for use in topical skin treatment formulations.

Preferably the carbanilide antibacterial agent used in the present invention has a minimum inhibitory concentration (MIC), at least against propionibacteria, of 20 μg/ml or less, more preferably 5 μg/ml or less, most preferably 1 μg/ml or less. More preferably the carbanilide also exhibits such characteristics in the presence of at least one of, preferably both of, lipid and sodium chloride, for instance as tested in the examples below.

Preferably the DAS or derivative used in the present invention has a minimum inhibitory concentration (MIC), at least against P. gingivalis, of 10 μg/ml or less, more preferably 5 or 2 or 1 μg/ml or less, such as from 0.1 to 2 μg/ml. Its corresponding minimum bactericidal concentration (MBC) is preferably 20 μg/ml or less, more preferably 10 μg/ml or less. Suitably the DAS or derivative also exhibits such characteristics in the presence of at least one of, preferably both of, saliva and gingival crevicular fluid, and/or in the presence of a component of such a fluid such as mucin or albumin—these are species which can be present in the mouth and hence performance in this context can be indicative of suitability for use in topical oral health care formulations.

Preferably the carbanilide antibacterial agent used in the present invention has a minimum inhibitory concentration (MIC), at least against P. gingivalis, of 10 μg/ml or less, more preferably 5 μg/ml or less, most preferably 2 or 1 μg/ml or less. Its corresponding minimum bactericidal concentration (MBC) is preferably 20 or 10 μg/ml or less, more preferably 5 or 2 or 1 μg/ml or less. More preferably the carbanilide also exhibits such characteristics in the presence of at least one of, preferably both of, saliva and gingival crevicular fluid, and/or in the presence of a component of such a fluid such as mucin or albumin.

MIC and MBC values may be measured using conventional assay techniques, for instance as described in the examples below.

The concentration of the DAS or derivative in the formulation might suitably be 0.1 or 0.2 or 0.5 w/w or greater, preferably 1 or 1.5% w/w or greater, more preferably 2 or 3 or 3.5 or 4% w/w or greater. Its concentration might be up to 15% w/w, preferably up to 10 or 9 or 8 or 7 or 6 or 5% w/w. For example, its concentration might be in the range from 0.1 to 10% w/w, or from 1 or 2 or 3 or 3.5 to 10% w/w or from 2 or 3 or 3.5 to 8% w/w, more preferably from 3 or 3.5 to 7% w/w or from 4 to 6% w/w, such as about 5% w/w.

In particular when the formulation is for use against a skin or skin structure condition such as acne, the concentration of the DAS or derivative may for example be from 1 to 10% w/w, or from 2 or 3 or 3.5 to 8% w/w. In particular when the formulation is for use against a condition affecting the oral cavity, the concentration of the DAS or derivative may be from 0.1 to 10% w/w, for example from 0.2 or 0.5 to 8 or 5% w/w. In particular when the formulation is for use against P. gingivalis, the concentration of the DAS or derivative may be from 0.05 to 5% w/w, for example from 0.05 or 0.1 to 5 or 2 or 1 w/w.

The concentration of the carbanilide antibacterial agent in the formulation might suitably be 0.01% w/v or greater, preferably 0.05 or 0.1% w/w or greater. Its concentration might be up to 5% w/w, preferably up to 2 or 1% w/w, more preferably up to 0.5 or 0.3 or 0.25 or 0.2% w/w. For example it might be present in the formulation at from 0.05 to 2% w/w, or from 0.05 to 1 or 0.5 w/w or in cases from 0.1 to 0.3% w/w, such as about 0.2% w/w.

In particular when the formulation is for use against a skin or skin structure condition such as acne, the concentration of the carbanilide may for example be from 0.05 to 2% w/w, or from 0.1 to 1 or 0.5% w/w. In particular when the formulation is for use against a condition affecting the oral cavity, the concentration of the carbanilide may be from 0.05 to 2% w/w, for example from 0.1 to 0.5 or 0.2% w/w.

Due to the presence of the other compound, it may be possible for the concentration of either the DAS or derivative or the carbanilide antibacterial agent, at the site of action when the formulation is applied in vivo, to be less than the MBC, or even than the MIC, of that compound alone. For instance the concentration of at least one of the compounds at this point may be 0.8 or less times its MBC or MIC, such as 0.5 or less, 0.25 or less or 0.125 or less.

Preferably the weight ratio of the DAS or derivative in the formulation to the carbanilide antibacterial agent, in particular when the formulation is for use in the treatment of a skin or skin structure condition such as acne, is from 1:50 to 50:1, or from 1:40 to 40:1 or from 1:30 to 30:1. It may be from 1:5 to 50:1 or from 1:5 to 40:1 or from 1:5 to 30:1. In particular when the formulation is to be used against propionibacteria, the weight ratio of the DAS or derivative to the carbanilide antibacterial agent may be from 1:1 or 2:1 to 50:1, or from 4:1 or 5:1 to 50:1, or from 10:1 to 40:1, or from 10:1 to 30:1, such as about 12.5:1 or 25:1.

In particular when the formulation is to be used for oral health care, more particularly against P. gingivalis and/or S. mutans, the weight ratio of the DAS or derivative to the carbanilide antibacterial agent may be from 50:1 to 1:50. The ratio may for example be 30:1 or below, or 25:1 or below. It may be up to 1:10, or up to 1:5 or 1:2 or 1:1. For example, the ratio may be from 30:1 or 25:1 to 1:1 or 3:1. Suitable ratios may for example be 25:1, 10:1, 5:1, 2.5:1 or 1:1. In particular when the formulation is to be used against P. gingivalis, the ratio may be from 10:1 to either 1:2 or 1:1, or from 5:1 to 1:1.

As described above, a formulation according to the invention is preferably suitable for, and more preferably adapted for, topical administration to human or animal, in particular human, skin. It may be suitable for, and more preferably adapted for, topical administration to the teeth, gums, gingivae, periodontium, tongue, skin or other tissue surfaces within the human or animal, in particular human, mouth. It may be suitable for, and/or adapted for, topical administration to the nares and/or tissue within the ears.

Such a formulation may take the form of a lotion, cream, ointment, varnish, foam, paste, gel, suppository or pessary or any other physical form known for topical administration, in particular a lotion, cream, ointment, varnish, foam, paste or gel. It may take the form of a solution or suspension, for instance for use as a disinfectant. It may comprise a formulation which is, or may be, applied to a carrier such as a sponge, swab, brush, tissue, cloth, wipe, skin patch, dressing (which includes a bandage, plaster, skin adhesive or other material designed for application to a tissue surface, in particular to a wound) or dental fibre to facilitate its topical administration. It may comprise a formulation which is, or may be, carried on or in an implant (including for example a chip for insertion into a periodontal pocket; or a dental filling, bridge or cap; or a denture), an impregnated dental fibre or denture or a chewing gum, tablet or chewable capsule. It may be intended for pharmaceutical (which includes veterinary but is preferably human) use, and/or for cosmetic or other non-medical care purposes (for example, for general hygiene or skin cleansing or for improving the appearance of the teeth or gums).

For use in the treatment of a condition affecting the oral cavity, the formulation may in particular take the form of a toothpaste, mouthwash, dentifrice, lozenge or buccal patch, or it may be carried in or on a dental fibre or tape or a denture.

The formulation may take the form of a nasal spray or of eye or ear drops.

The vehicle in which the DAS or derivative and the carbanilide antibacterial agent are contained may be any vehicle or mixture of vehicles which is suitable for topical application; the type chosen will depend on the intended mode and site of application. Many such vehicles are known to those skilled in the art and are readily available commercially. In the context of formulations for topical application to the skin, examples may for instance be found in Williams' “Transdermal and Topical Drug Delivery”, Pharmaceutical Press, 2003, and other similar reference books. See also Date, A. A. et al, Skin Pharmacol. Physiol., 2006, 19(1): 2-16 for a review of topical drug delivery strategies.

In the context of formulations for topical application within the oral cavity, examples of suitable vehicles may be found in “Oral Hygiene Products and Practice”, 1988, Morton Prader, Ed., Marcel Dekker, Inc., New York, N.Y., USA.

In the context of formulations for ocular use, examples of suitable vehicles may be found in “Ocular Therapeutics and Drug Delivery: A Multidisciplinary Approach”, 1995, Indra K. Reddy, Ed., CRC Press; and “Ophthalmic Drug Delivery Systems”, 2003, Ashim K. Mitra, Ed., Informa Healthcare.

Either or both actives may be present in the form of a suspension or other type of multi-phase dispersion, as described above.

Also as described above, the vehicle may be such as to target a desired site and/or time of delivery of the formulation. It may for instance target the formulation to the skin or hair follicles or to the anterior nares (the latter being particularly suitable when the formulation is used as a prevention treatment against staphylococci) not preferably to the skin or hair follicles. It may target the formulation to the gums or teeth or other areas within the oral cavity. It may delay or otherwise control release of the formulation over a particular time period. Either or both of the DAS or derivative and the carbanilide antibacterial agent may be microencapsulated, for instance in liposomes—particularly suitable liposomes, for topical application to the skin, are those made from stratum corneum lipids, eg, ceramides, fatty acids or cholesterol.

In some cases a polar vehicle may be preferred. Where the formulation is intended for use on the skin, the vehicle may be primarily non-aqueous, although in the case of an anti-acne treatment an aqueous vehicle may be used. The vehicle may be surface-active, in particular when it is intended for use in treating surfaces, for instance to cleanse instruments or working areas in particular against staphylococci. It is suitably volatile. In cases the vehicle may be alcohol-based or silicon-based.

By way of example, a lotion or gel formulation according to the invention, in particular one intended for application to the skin, may contain a mixture of water, an alcohol such as ethanol or phenoxyethanol and a glycol such as propylene glycol.

In an embodiment of the invention, the formulation containing the DAS or derivative and the carbanilide antibacterial agent also contains:

(i) a polyoxyalkylene-based solubilising agent;

(ii) an organic solvent, in particular an alcohol;

(iii) a thickening agent; and

(iv) water.

It has surprisingly been found that this particular combination of ingredients can yield a stable formulation despite the typically low solubility of carbanilides such as TCC in aqueous solvent mixtures, and even at higher concentrations of the DAS active than have previously been thought feasible (for example 3.5% w/w or greater, such as from 3.5 to 8% w/w). To our knowledge, DASs and their derivatives have not previously been formulatyed at such high concentrations, in particular for topical use.

In an embodiment of the invention, the formulation is in the form of a gel. It may for example have a viscosity of from 25,000 to 300,000 cps, or from 50,000 to 200,000 cps.

In a formulation of the type defined above, the solubilising agent (i) is polyoxyalkylene-based, for example polyoxyethylene (polyethylene glycol, PEG)-based. It may comprise a derivative of a polyoxyalkylene, for example an ether or more particularly an ester. It may for example be selected from (a) polyalkoxylated esters, other than polyalkoxylated sorbitan esters and (b) polyalkoxylated alkyl ethers. More particularly it may be selected from (a) polyethoxylated esters, other than polyethoxylated sorbitan esters and (b) polyethoxylated alkyl ethers. It is suitably nonionic.

A polyalkoxylated ester may be a polyalkoxylated fatty acid ester. The fatty acid component may be a C₁₀ to C₂₀ group or a C₁₂ to C₁₈ group, for example stearic acid (C₁₈) or 12-hydroxystearic acid. It may in particular be a hydroxyl-substituted fatty acid group. Suitable such solubilising agents are commercially available as Myrj™ 45 (PEG 400-monostearate) and Solutol® HS 15 (PEG-15-hydroxystearate, also known as PEG 660 12-hydroxystearate and Macrogol-15-hydroxystearate).

Solutol® HS 15 is a nonionic solubilising agent available from BASF AG. It consists primarily of polyglycol mono- and di-esters of 12-hydroxystearic acid (the lipophilic part of the molecule) and of about 30% of free polyethylene glycol (the hydrophilic part). It is prepared by reacting 15 moles of ethylene oxide with one mole of 12-hydroxystearic acid.

Solubilisers of this general type are known for use in injectable and in cases oral pharmaceutical formulations, but are not widely used in, or recommended for use in, formulations intended for topical application. There are thus few available data regarding appropriate concentrations for such agents in topical formulations.

A polyalkoxylated ester may be a polyalkoxylated glyceryl ester (also known as a polyalkoxylated glyceride), which is a glyceride ester of a polyalkylene glycol (typically PEG). It may contain mono-, di- or tri-glycerides, partial glycerides or mixtures thereof. It may for example be formed by esterification of a polyalkylene glycol with a glyceride oil of an appropriate chain length. The glyceride components may for example contain from 6 to 20 or from 8 to 18 carbon atoms. Suitable such solubilising agents are commercially available as Glycerox™ and Labrasol™ Glycerox™ esters, for example, are PEG-based products which are commercially available from Croda Inc in a number of grades reflecting the molecular weight of the PEG, for example PEG-6, PEG-7, PEG-8 (L8) and PEG-15 (L15). Of these, the PEG-6 version may be preferred. Glycerox™ esters are sold for use as emollients, for example in bath products, skin cleansers and shampoos, and as solubilisers, superfatting agents, dispersing agents and emulsifiers.

A glyceride solubilising agent may for example be Glycerox™ 767 (polyoxyethylene (6) glyceryl monocaprylate/caprate, a mixture of caprylic and capric glycerides of PEG-6) or Glycerox™ HE, which is a PEG-7 glyceryl cocoate.

A polyalkoxylated alkyl ether may be a polyalkoxylated ether of a fatty alcohol such as a C₁₂ to C₂₀ or C₁₄ to C₂₀ alcohol, for example cetyl (C₁₆), stearyl (C₁₈) or oleyl (C_{18:1}). It may in particular be a polyethoxylated alkyl ether, also known as a macrogol ether. Suitable such solubilising agents are commercially available as Brij™ 97 (PEG-monooleyl ether) and Cremophor™ A 25 (a mixture of stearic and cetyl ethers).

In general, the polyalkylene glycol element of a solubilising agent used in the present invention may have any appropriate molecular weight.

The solubilising agent (i) may in particular be a polyethoxylated ester, more particularly a fatty acid ester or a polyethoxylated glyceride. Solutol® HS 15 and Glycerox™ 767, in particular the former, have been found to be suitable solubilising agents for use in the present invention.

The organic solvent (ii) used in the formulation of the invention is suitably an alcohol, for example methanol, ethanol, isopropanol or phenoxyethanol. It may be selected from methanol, ethanol, isopropanol and mixtures thereof, preferably from methanol, ethanol and mixtures thereof. In an embodiment of the invention the organic solvent is ethanol.

The thickening agent(iii) may be any thickening agent suitable for topical application. It is suitably a gelling agent. It may in particular be a cellulose-based thickening agent such as ethyl cellulose, hydroxyethyl cellulose, hydroxypropyl cellulose, hydroxypropyl methyl cellulose or a carboxymethyl cellulose. Again such agents may be used in the form of a (preferably pharmaceutically acceptable) salt such as for instance the sodium salt. The thickening agent may be a polymeric thickening agent such as a carbomer, which will typically be a homopolymer of acrylic acid, cross-linked with an allyl ether.

A preferred thickening agent is hydroxyethyl cellulose.

The concentrations of the active ingredients, ie, the DAS or derivative and the carbanilide antibacterial agent, may be as described above.

The solubilising agent is preferably present at a concentration of 10% w/w or greater, more preferably 15 w/w or greater. It may be present at a concentration of up to 50% w/w, suitably up to 40 or 30 or 25 w/w. Its concentration may for example be from 10 to 30% w/w or from 15 to 25 w/w, such as about 20% w/w.

The organic solvent is preferably present at a concentration of 10% w/w or greater, more preferably 15 or 17% w/w or greater. It may be present at a concentration of up to 30% w/w, suitably up to 25% w/w. Its concentration may for example be from 10 to 30% w/w or from 15 to 25 or 30% w/w, such as about 20% w/w.

The thickening agent is preferably present at a concentration of 0.2% w/w or greater, more preferably 0.5 w/w or greater. It may be present at a concentration of up to 5% w/w, suitably up to 3% w/w. Its concentration may for example be from 0.5 to 5% w/w or from 0.5 to 3% w/w, such as about 1 w/w.

According to a preferred embodiment, the invention therefore provides an antibacterial formulation which contains:

(i) from 2 to 8% w/w of a dialkyl sulphosuccinate or derivative thereof;

(ii) from 0.05 to 0.5 w/w of a carbanilide antibacterial agent;

(iii) from 10 to 30% w/w of a polyoxyalkylene-based solubilising agent;

(iv) from 10 to 30% w/w of an organic solvent, in particular an alcohol;

(v) from 0.5 to 5 w/w of a thickening agent; and

(vi) the balance water.

It has surprisingly been found that formulations of these types can be stable even when containing both a DAS or derivative thereof and a carbanilide antibacterial agent such as TCC, and moreover when containing higher concentrations of the DAS or derivative than have previously been thought feasible.

DAS- and carbanilide-containing formulations of the type provided by the invention are by no means, in the context of their intended use, straightforward combinations of standard excipients. The use of an alcohol/water mixture alone, for example, could be insufficient to solubilise a carbanilide active such as TCC. It has been found, however, that of the many commonly available solubilising agents, only a small number can be successfully used to formulate DASs and carbanilides into stable topical gels, in particular at the relatively high DAS concentrations preferred in the present invention. The more conventional poloxamer solubilisers such as those in the Tween™ series, for example, have been found not always to be suitable for use in such formulations. The use of solubilising agents such as Solutol® HS 15, which is more conventionally used in aqueous parenteral formulations rather than topical ones, is also far from intuitive in the context of a topical gel formulation and at the relatively high concentrations found to be preferred in a formulation according to the invention.

Particularly surprising is the fact that in the absence of the DAS, the remaining ingredients of the formulation appear not always to form a stable mixture. There therefore appears to be a benefit, in terms of the stability of the overall formulation, due to the use of the invented combination of ingredients, which effect could not have been predicted from the prior art.

Generally speaking a formulation according to the invention may contain standard excipients and other additives known for use in pharmaceutical or veterinary formulations. For example, where the formulation is intended for topical application to the skin, in particular to treat skin and skin structure conditions and/or to treat conditions such as acne or atopic dermatitis, examples of suitable excipients and additives include emollients, perfumes, antioxidants, preservatives, stabilisers, gelling agents and surfactants; others may be found in Williams' “Transdermal and Topical Drug Delivery”, supra. For the treatment of acne, however, it may be preferred for the formulation not to contain an emollient.

Such a formulation may further contain additional active agents such as antimicrobial (in particular antibacterial) agents. For example, it may contain one or more agents selected from anti-acne agents, keratolytics, comedolytics, anti-inflammatories, anti-proliferatives, antibiotics, anti-androgens, sebostatic agents, anti-pruritics, immunomodulators, agents which promote wound healing, antimicrobial agents and mixtures thereof; it may instead or in addition contain one or more agents selected from sunscreens, moisturisers, emollients and mixtures thereof.

An additional antimicrobial agent may for example be selected from the group consisting of biocides, disinfectants, antiseptics, antibiotics, bacteriophages, enzymes, anti-adhesins, immunoglobulins and mixtures thereof; it is preferably active as a bactericide, in particular against propionibacteria.

It may however be preferred for the DAS or derivative and the carbanilide antibacterial agent to be the only active agents in the formulation, or at least to be the only antimicrobially or antibacteriallyactive agents and/or the only anti-acne active agents.

In the case where the formulation is intended for topical application within the oral cavity, suitable excipients and additives include those known for use in oral health care formulations. Examples include flavourings, antioxidants, preservatives, stabilisers, gelling agents and surfactants; others may be found in “Oral Hygiene Products and Practice”, 1988, supra. Surfactants may help to disrupt, and/or prevent formation of, microbial biofilms.

Such a formulation should contain an orally acceptable and systemically non-toxic vehicle. For example, where it takes the form of a toothpaste, a typical vehicle might include water and a humectant to provide a liquid base, together with one or more of a thickener, a surfactant and a polishing agent. Suitable humectants include glycerol, sorbitol and polyethylene glycol, and in particular mixtures thereof. A polyethylene glycol humectant may for example have a molecular weight range of from 200 to 1000 or from 400 to 800.

Suitable thickeners for use in toothpaste formulations include natural and synthetic gums and colloids such as carrageenan, xanthan gum and sodium carboxymethyl cellulose, as well as gum tragacanth; starch; polyvinyl pyrrolidone; cellulosic thickeners such as hydroxyethyl propyl cellulose, hydroxybutyl methyl cellulose, hydroxypropyl methyl cellulose, hydroxyethyl cellulose and water soluble salts of cellulose ethers such as sodium carboxymethyl cellulose or sodium carboxymethyl hydroxyethyl cellulose; and carboxyvinyl polymers. Suitable inorganic thickeners include colloidal silica, colloidal magnesium aluminium silicate, finely divided silica and synthetic hectorite. Mixtures of thickeners may also be used.

Suitable surfactants for use in toothpaste formulations according to the invention, in addition to the DAS or derivative, include water soluble detergents. In general they may be anionic, nonionic, cationic, zwitterionic, amphoteric or ampholytic, but are preferably anionic. Examples of suitable anionic surfactants include higher alkyl sulphates such as sodium lauryl sulphate, and higher fatty acid esters of 1,2 dihydroxy propane sulphonate. Examples of suitable water soluble nonionic surfactants include the polymeric condensation products of hydrophilic alkylene oxide group-containing compounds (typically ethylene oxide) with organic hydrophobic compounds (for example those having aliphatic chains of about 12 to 20 carbon atoms). Such products include the “ethoxamers” and include for example the condensation products of poly(ethylene oxide) with fatty acids, fatty alcohols, fatty amides and other fatty moieties, as well as with propylene oxide and polypropylene oxides (the latter being available, for example, under the trade name Pluronic®). In cases however it may be preferred to avoid the inclusion of additional surfactants in a formulation according to the invention.

A toothpaste will suitably contain an abrasive or polishing agent. Suitable such agents include siliceous materials (including gels and precipitates, such as precipitated amorphous hydrated silicas, aluminium silicate, zirconlure silicate, silica gel and colloidal silica); carbonates and bicarbonates such as calcium carbonate and sodium bicarbonate; phosphates such as sodium metaphosphate, potassium metaphosphate, tricalcium phosphate, dicalcium orthophosphate dehydrate, calcium phosphate dihydrate, anhydrous dicalcium phosphate, calcium pyrophosphate, calcium polymetaphosphate, magnesium orthophosphate, trimagnesium phosphate and insoluble sodium polymetaphosphate; alumina trihydrate; calcined alumina; bentonite; complex amorphous alkali metal aluminosilicates; and resinous abrasive materials such as particulate condensation products of urea and formaldehyde. Others are disclosed in U.S. Pat. No. 3,070,510. Mixtures of such polishing agents may also be used. The abrasive or polishing agent should not excessively abrade tooth enamel or dentin.

Silica abrasive agents may be particularly preferred for use in the present invention.

Where a formulation according to the invention takes the form of a mouthwash or dentifrice, it may for example contain a water/alcohol (eg, water/ethyl alcohol) solution and optionally one or more other ingredients selected for example from flavourings, sweeteners, humectants, surfactants, emulsifiers if necessary and mixtures thereof. Suitable humectants include those described above, in particular glycerol and sorbitol. One or more additional antimicrobial agents may also be included.

Non-soap surfactants (for example nonionic, cationic or amphoteric surfactants) may be used in mouthwash formulations. Suitable nonionic surfactants include the condensation products of hydrophilic alkylene oxide group-containing compounds with organic hydrophobic compounds, as described above. Other suitable nonionic synthetic detergents include: the polyethylene oxide condensates of alkyl phenols; those derived from the condensation of ethylene oxide with the product resulting from the reaction of propylene oxide and ethylene diamine, the condensation products of aliphatic alcohols having from 8 to 18 carbon atoms with ethylene oxide; and the polyoxyethylene derivatives of fatty acid partial esters of sorbitol anhydride (for example the commercially available Tween® products).

Suitable cationic detergents include quaternary ammonium compounds, in particular those having one long alkyl chain of about 8 to 18 carbon atoms, for example lauryl trimethylammonium chloride, cetyl pyridinium chloride, cetyl trimethylammonium bromide, di-isobutylphenoxyethyldimethylbenzylammonium chloride, coconutalkyltrimethylammonium nitrite, cetyl pyridinium fluoride and the like.

Suitable amphoteric detergents include derivatives of aliphatic secondary and tertiary amines in which one of the aliphatic substituents contains from about 8 to 18 carbon atoms and one contains an anionic water solubilising group such as carboxylate, sulphate, sulphonate, phosphate or phosphonate.

Other suitable surfactants, for use in formulations according to the invention, may be found in McCutcheon's Detergents and Emulsifiers and in U.S. Pat. No. 4,051,234. Again, however, it may in certain cases be preferred to avoid additional surfactants.

A formulation according to the invention, when intended for topical application within the oral cavity, may contain one or more agents selected from abrasives, bleaching agents, tooth whitening agents (eg, peroxides or sodium perborate), surface active agents/detergents as described above, foaming agents, sources of fluoride ions or fluorine-containing ions, zinc salts, non-cariogenic sweeteners such as saccharin or aspartame or dextrose or levulose, other flavourings such as peppermint or spearmint or aniseed, menthol, desensitising agents, anti-tartar/sequestering agents or anti-calculus agents (for example metal salts such as zinc chloride, zinc acetate or zinc oxide; pyrophosphate salts such as alkali metal or ammonium pyrophosphates; or diphosphonates), sodium bicarbonate, anionic polycarboxylates, enzymes such as lactoperoxidases, humectants as described above, binders such as carboxyvinyl polymers, pH regulating buffers, preservatives, colours/dyes (for example chlorophyll or titanium dioxide), plant extracts, anti-plaque agents, additional antimicrobial (for example antifungal or antibacterial, especially antibacterial) agents, anaesthetics, and mixtures thereof.

In this context an additional antimicrobial agent may be selected from the group consisting of biocides, disinfectants, antiseptics, antibiotics, bacteriophages, enzymes, anti-adhesins, immunoglobulins and mixtures thereof; it is preferably active as a bactericide, in particular against S. mutans and/or P. gingivalis and/or one or more other bacteria associated with oral health problems.

Again however it may be preferred for the DAS or derivative and the carbanilide antibacterial agent to be the only active agents in the formulation, or at least to be the only antimicrobially or antibacterially active agents and/or the only agents active against P. gingivalis.

Suitable sources of fluoride or fluorine-containing ions are water soluble fluorides such as water soluble alkali metal or alkaline earth metal fluorides, for example sodium, potassium and barium fluorides (in particular alkali metal fluorides); copper fluorides, such as cuprous fluoride; tin fluorides; fluorosilicates such as sodium or ammonium fluorosilicate; fluorozirconates such as sodium or ammonium fluorozirconate; monofluorophosphates such as sodium or potassium monofluorophosphate; mono-, di- and tri-aluminium fluorophosphates; and fluorinated pyrophosphates such as fluorinated sodium calcium pyrophosphate.

In the case where the formulation is intended for use in the treatment of body odour, it will suitably contain an anti-perspirant such as an aluminium or aluminium-zirconium salt, and/or a deodorising agent. It may be in the form of an aerosol, or of a roll-on or “stick” deodorant of known type, containing appropriate conventional liquid or solid carriers and excipients. It may contain one or more perfumes.

In the case where the formulation is intended for use in the treatment of an ocular infection, it may take the form of a cream or ointment, or of eye drops, or of an eye rinse. Such formulations are typically aqueous based, and may contain conventional excipients, including thickeners where appropriate.

In the case where the formulation is intended for application to a non-living area or surface, for instance as a disinfectant, it may take the form of a solution or suspension of the DAS or derivative and the carbanilide antibacterial agent in an appropriate fluid vehicle such as an alcohol or a water/alcohol mix. Again conventional excipients and other additives may be included, as may one or more additional antimicrobial (in particular antibacterial) agents.

In particular when the formulation is for use in controlling the transmission of a bacterial infection, it may be in the form of a skin wash (for example a hand wash), or of a surface disinfectant such as a spray, or of a cleansing fluid for example for use in disinfecting dentures or surgical (including dental) instruments. It may be carried in or on a cloth, wipe, brush or other cleaning utensil, or a substrate such as a preparation surface or implement or a packaging material; in such cases an item may be impregnated with, or coated with, the formulation.

Generally speaking a formulation according to the invention may contain one or more agents which enhance the activity of another active agent present in the formulation, or reduce a side effect of such an active, or improve patient compliance on administration of the formulation. It may contain one or more agents which facilitate penetration of an active agent, in particular the DAS or derivative and/or the carbanilide antibacterial agent, into microbial biofilms. It may contain one or more agents which control the site and/or rate of release of an active agent following administration.

It may be preferred for a formulation according to the invention not to include benzyl alcohol. Benzyl alcohol is commonly used as a preservative in topical pharmaceutical formulations, but in some cases has been found to be determimental to the stability of a formulation according to the present invention.

In cases it may be preferred for a formulation according to the invention not to contain a laureth sulphosuccinate, in particular disodium laureth sulphosuccinate.

In cases it may be preferred for a formulation according to the invention not to contain a primary ethoxylated alcohol, for example of the type disclosed in U.S. Pat. No. 5,977,049.

In cases it may be preferred for a formulation according to the invention not to contain a water soluble polyethylene glycol, particularly of molecular weight at least about 200, for example as disclosed in U.S. Pat. No. 5,922,768.

In cases it may be preferred for a formulation according to the invention not to contain an amphoteric surfactant, for example of the type disclosed in U.S. Pat. No. 5,883,059.

A formulation according to the invention may be suitable for, more preferably adapted for, use in an area or on a surface other than living tissue, for instance to treat floors or walls (whether internal or external), work surfaces or instruments, to disinfect dentures or to cleanse hair or nails so as to reduce bacteria levels. It may be suitable for application to non-living tissue (for instance for use as a preservative) or clothing (for instance for bio-agent decontamination). In these cases the excipients, vehicles and/or other additives included with the DAS or derivative and the carbanilide antibacterial agent may be different to those included in a topical skin care or oral health care formulation, but again may be conventional as known for use in such contexts. In particular the formulation, or either or both of the two active agents, may be provided in the form of a concentrate which can be diluted to a suitable concentration (for example as described above) prior to use.

A formulation according to the invention may be incorporated into, and hence applied in the form of, another product such as a cosmetic, a skin or hair care preparation (for example a skin cleanser, toner or moisturiser, or a shampoo, conditioner, styling mousse or gel or hair spray), a deodorant or anti-perspirant, a dental or other oral health care-preparation (for example a toothpaste, mouthwash, dentifrice, dental gel or dental floss, in particular a toothpaste or mouthwash), a cleansing preparation (for example a hand wash for use by surgeons prior to treating patients), a pharmaceutical (which, includes veterinary) preparation, a cosmeceutical preparation, a toiletry product (for instance a bath or shower additive or a soap), a laundry or other fabric treatment product or an agricultural or horticultural product.

The invention thus provides, according to a second aspect, a product which incorporates an antibacterial formulation according to the first aspect.

A formulation according to the invention may be marketed with an indication that it has antibacterial activity, or enhanced antibacterial activity, for example against one or more of the pathogens referred to herein. The marketing of such a formulation may for example include an activity selected from (a) enclosing the formulation in a container or package that comprises the relevant indication; (b) packaging the formulation with a package insert that comprises the indication; (c) providing the indication in a publication that describes the formulation; and (d) providing the indication in a commercial which is aired for instance on the radio, television or internet. The antibacterial activity of the formulation may be attributed, in such an indication, at least partly to the presence of the combination of the DAS or derivative and the carbanilide antibacterial agent.

The invention may involve assessing the antibacterial activity of the formulation during or after its preparation, for instance against one or more of the pathogens referred to herein. It may involve assessing the antibacterial activity of the formulation both before and after incorporation of the DAS or derivative and/or the carbanilide antibacterial agent, for example so as to confirm that either or preferably both contribute to the antibacterial activity of the formulation.

The formulation of the invention may be prepared in situ, at or immediately before its point of use, for instance its application to the skin or another surface. Thus according to a third aspect, the present invention provides a kit for preparing an antibacterial formulation, such as a formulation according to the first aspect, the kit comprising a source of a DAS or derivative thereof and a source of a carbanilide antibacterial agent, together with instructions for combining the two compounds so as to make the formulation at or before the point of intended use, and/or for the co-administration of the two compounds to a surface such as the skin. The two compounds may each be present in a suitable respective vehicle.

According to one embodiment, the formulation or kit of the invention may contain both a DAS or derivative thereof and a carbanilide antibacterial agent, each encapsulated (for instance microencapsulated) in a separate delivery vehicle; this might for instance allow their release, and hence their contact with one another, only at the intended site of administration.

A fourth aspect of the invention provides a method for preparing an antibacterial formulation, which method involves mixing together a DAS or derivative thereof and a carbanilide antibacterial agent, preferably together with a pharmaceutically acceptable vehicle.

According to a fifth aspect of the invention there is provided a formulation (preferably a formulation according to the first aspect of the invention) containing a DAS or pharmaceutically acceptable derivative thereof and a carbanilide antibacterial agent, for use in the treatment of a condition affecting the human or animal body, which condition is caused by, transmitted by and/or exacerbated by (in particular either caused or transmitted by) bacterial activity. The condition may be for example a skin, skin structure, oral, ocular, aural, nasal, soft tissue or vaginal condition.

For the purposes of the fifth and subsequent aspects of the invention, the two alkyl groups of the DAS or derivative are suitably C₁ to C₁₂ alkyl groups, more suitably C₂ to C₁₀, such as C₆ to C₁₀ and in particular C₈.

In the context of the present invention, treatment of a condition encompasses both therapeutic and prophylactic treatment, of either an infectious or a non-infectious condition, in either a human or animal but in particular a human. It may involve complete or partial eradication of the condition, removal or amelioration of associated symptoms, arresting subsequent development of the condition, and/or prevention of, or reduction of risk of, subsequent occurrence of the condition. It will typically involve use of the DAS or derivative and the carbanilide as a bactericidal combination. It may involve the prophylactic treatment of any area of the body, in particular the skin or nares or another epithelial or mucosal surface, against bacterial infections, including against staphylococcal infections such as those associated with MRSA.

The treatment of a condition also embraces the prevention, or reduction of risk of, dissemination or transmission of the condition, for example from person to person. In this context, the DAS or derivative and the carbanilide antibacterial agent may be used in combination as a disinfectant against the relevant bacterium, for example for antisepsis of the skin and/or other appropriate parts of the body, or for the general disinfection of surfaces in an area believed to be contaminated with, or at risk of contamination with, the bacterium. Thus the invented combination may be used to treat an outbreak of a particular pathogen, for example a nosocomial pathogen such as S. aureus (including resistant strains such as MRSA, VISA or GISA), E. faecalis or C. difficile.

In the context of the present invention, treatment of a condition may in particular involve use of the formulation against propionibacteria, and/or against Gram-positive cocci such as staphylococci or streptococci, and/or against bacteria associated with an infection within the oral cavity, and/or against bacteria associated with body odour.

It may involve use of the formulation against bacterial biofilm formation. Thus, the formulation may be used to treat a condition which is caused, transmitted and/or exacerbated by (in particular caused or transmitted by) bacterial biofilm formation, in particular biofilm formation which is caused or exacerbated by, or which otherwise involves (in particular which is caused by), P. gingivalis.

In an embodiment of this fifth aspect of the invention, the formulation is for use against one or more bacteria associated with skin or skin-borne infections. It may be for use against Gram-positive bacteria, for example staphylococci and/or propionibacteria, in particular against strains of Propionibacterium acnes. In an embodiment, it is for use against one or more bacteria associated with acne, such as P. acnes and in some instances P. granulosum.

According to an embodiment of the fifth aspect of the invention, the formulation is for use in the treatment of a skin or skin structure condition. Such a condition may be a primary or secondary infection. It may for example be a superficial or uncomplicated skin infection amenable to local therapy. It may be acne or an infection associated with acne. It may be a primary or secondary infection due to S. aureus (including MRSA) or a group a beta haemolytic streptococcus (S. pyogenes).

The formulation may in particular be for use in the treatment of acne (ie, as an anti-acne agent).

Skin and skin structure conditions which might be treated according to the invention include acne, infected atopic eczema, superficial infected traumatic lesions, wounds, burns, ulcers, folliculitis, mycoses and other primary and secondary skin and skin structure infections. In particular the invented formulation may be for use in treating acne or acne lesions (for instance, to reduce acne-related scarring).

Acne is a multifactorial disease of the pilosebaceous follicles of the face and upper trunk, characterised by a variety of inflamed and non-inflamed lesions such as papules, pustules, nodules and open and closed comedones. Its treatment can therefore encompass the treatment (which embraces prevention or reduction) of any of these symptoms, and references to use as an anti-acne agent may be construed accordingly.

In particular, the treatment of acne encompasses the treatment (including prevention) of lesions and/or scarring associated with acne. It also encompasses the treatment of a propionibacterial infection and/or the inhibition of propionibacterial activity which could cause or be otherwise associated with acne or its symptoms.

In general, the present invention will be used for the treatment of symptoms which are directly due to acne rather than for instance infections which may arise as a consequence of treating acne with other actives such as antibiotics, and/or secondary infections caused by opportunistic pathogens, which can arise in skin already affected by acne.

Thus, in general terms the invention can provide a formulation containing a DAS or derivative and a carbanilide antibacterial agent, for use in the treatment of acne.

Atopic dermatitis or eczema (atopic eczema and dermatitis syndrome AEDS), which may also be treated using the present invention, can frequently become infected by Gram-positive bacteria, most commonly by Staphylococcus aureus (David T J, 1989 Journal of the Royal Society of Medicine 82: 420-422) but also by members of the genus Streptococcus (Brook I, 2002 Journal of Medical Micrbiology 51: 808-812). According to the fifth aspect of the invention, the formulations may be use in the treatment of infected atopic dermatitis since such combinations of actives have been shown to be active against S. aureus (including Methicillin Resistant. S. aureus (MRSA), Epidemic Methicillin Resistant S. aureus (EMRSA), Vancomycin-Intermediate Resistant S. aureus (VISA) and Glycopeptide-Intermediate Resistant S. aureus (GISA)) and Streptococcus pyogenes. The invented formulation may be for use against one or more such bacteria.

Human skin is susceptible to infection by a wide range of Gram-positive bacteria. These conditions include but are not restricted to folliculitis, boils and carbuncles, impetigo most usually caused by Staphylococcus aureus and other infections including erysipelas caused by members of the genus Streptococcus, erythrasma and cellulitis caused by both staphylococci and streptococci. Other Gram-positive bacteria may also be involved in these infections including members of the genera Bacillus, Clostridium and Enterococcus. According to the fifth aspect of the invention, the invented formulation may be for use in the treatment of infected dermatoses, skin infections, superficial infected wounds and soft tissue infections since such combinations of actives have been shown to be active against S. aureus, including MRSA, EMRSA, VISA and GISA, as well as against Streptococcus pyogenes, members of the genus Enterococcus (including E. faecalis) and members of the genus Bacillus and Clostridium.

Infected dermatoses, skin infections, superficial infected wounds and soft tissue infections may also include polymicrobial infections of the skin that are caused by both Gram-positive and Gram-negative bacteria. In such cases the invented formulation may be used in combination with an additional antimicrobial agent, for example either a topical or a systemic agent which is active against Gram-negative bacteria.

In an embodiment of the fifth aspect of the invention, the invented formulation may be for use as a treatment against staphylococci, which might otherwise cause for example MRSA-associated infections. It may be for use as a treatment against staphylococci on the skin, or in the nares, eyes, or ears. The formulation may in particular be for use in a prophylactic treatment against staphylococci (in particular S. aureus) in the nasal carriage.

Approximately 25 to 30% of healthy individuals carry Staphylococcus aureus in the nares. The organism is also carried at other body sites and at higher prevalence in predisposed individuals such as those with atopic dermatitis. Antibiotic-resistant strains of Staphylococcus aureus (eg, MRSA) are also widely distributed both in the hospital environment and in the community. These factors contribute to the risk of nosocomial S. aureus infections especially in patients undergoing surgery (Grundmann H, Aires-de-Sousa M, Boyce J, Tiemersma E, 2006 Lancet 368: 874-85; Herwaldt L A, 2003 Surgery 134(5 Suppl):S2-9). Combinations of DASs and carbanilides have been shown capable of activity against S. aureus and may be used prophylactically to eradicate and/or prevent colonisation of the nares and skin by this organism. This can be used for example in patients and hospital staff to prevent infections caused by S. aureus. The combinations are particularly well suited for this purpose as they can be active against antibiotic-resistant strains of S. aureus including MRSA, EMRSA, VISA and GISA.

According to a further embodiment of the fifth aspect of the invention, the invented formulation is for use in the treatment (which includes prevention) of body odour, for example in the axilla or feet. It may thus be for use against the bacteria implicated in this condition, in particular aerobic diphtheroids of the genus Corynebacterium.

Human body odour is formed by the action of commensal skin bacteria on the odourless secretions of sweat glands. For example the action of members of the genus Corynebacterium have been shown to release the odiferous compounds 3-hydroxy-3-methylhexonic acid and 3-hydroxy-2-methylhexonic acid from odourless precursors (Natsch A, Gfeller H, Gygax P et al, 2003 Journal of Biological Chemistry 278 (8): 5718-5727). The invented formulation may thus be for use in the prevention of body odour as such combinations of compounds have been shown to be active against species of the genus Corynebacterium, including corynebacteria of human origin such as C. mucifaciens, C. jeikeium, C. striatum and C. xerosis. Such combinations have also been shown to be active against other members of the bacterial human skin microflora such as cutaneous propionibacteria, and coagulase negative staphylococci that may also contribute to human body odour. According to the fifth aspect of the invention, the formulation may therefore be used against one or more such bacteria in cases it may be used against micrococci and/or related species, for Micrococcus luteus.

In an embodiment of the fifth aspect of the invention, the invented formulation is for use in the treatment of a bacterial condition within the oral cavity. It may therefore be for use against one or more bacteria associated with conditions affecting the oral cavity, for example dental caries, periodontal diseases such as gingivitis and periodontitis, or halitosis. It may be used for the creation and/or maintenance of fresh-smelling breath. It may be used for the treatment of mouth ulcers.

Thus for example, the invented formulation may be for use against strains of P. gingivalis. Fuithermore, such combinations of compounds have been found to be active against Gram-positive and Gram-negative bacteria known to be associated with dental plaque and the formation of biofilms which have been implicated in the etiology of periodontal diseases, including gingivitis. The formulation may therefore be of use against, among others, the following Gram-positive bacteria: Streptococcus mutans, Streptococcus sobrinus and Actinomyces naeslundii, and surprisingly the following Gram-negative bacteria: Porphyromonas gingivalis and Prevotella nigrescens, all of which can contribute to the microflora found in dental biofilms involved in periodontal disease.

The formulation may be for use in the treatment of a periodontal disease, and/or as an anti-plaque agent.

Periodontal diseases which may be treated using the present invention (the term “treat” embracing both therapeutic and prophylactic measures, as defined above) include for example dental plaque-induced gingival diseases; chronic (previously adult) periodontitis; aggressive periodontitis (formerly early-onset, prepubertal, juvenile or rapidly progressive periodontitis); necrotising periodontal diseases; abcesses of the periodontium; and post-operative bacterial infections (in particular those which are caused, transmitted and/or exacerbated by P. gingivalis). The invention may also be used to treat microbial infections (in particular those which are caused, transmitted and/or exacerbated by P. gingivalis) of wounds other lesions within the oral cavity, including those arising due to other medical conditions such as oral candidiasis.

Associated symptoms which may be treated using the invention include mouth ulcers, dental pain, discomfort, inflammation, bleeding, pus secretion, halitosis, tooth mobility, tooth loss, swelling or inflammation caused by any of the foregoing.

Since plaque formation on tooth surfaces can also cause, exacerbate or accompany periodontal diseases, in accordance with the fifth aspect of the present invention the formulation may be used to reduce or prevent plaque formation, and/or to alter (suitably beneficially) the bacterial composition of plaque.

It has further been found that periodontal inflammatory diseases may be linked to (ie, may in cases cause, increase susceptibility to and/or exacerbate) other more serious, often more systemic conditions. For example, periodontal diseases, and/or the by-products generated by associated pathogens such as P. gingivalis, have been linked with coronary artery disease and other cardiovascular diseases such as myocardial infarction, atherosclerosis and angina, as well as with conditions associated with arterial inflammation or blood clot formation and with an increased risk of pre-term low-weight births (Gotsman et al, J. Periodontol. May 2007, 78(5): 849-858; Noack et al, J. Periodontol. 2001, 72: 1221-1227; Seymour et al, Clinical Microbiology and Infection, 13 (Suppl 4): 3-10). The present invention may accordingly be used, indirectly,.to treat any such condition as well as to treat infections within the oral cavity and associated periodontal diseases.

It has also been recognised that periodontal diseases can pose a threat to the health of those suffering from chronic diseases such as diabetes, respiratory diseases, osteoporosis and AIDS (Kuo et al, Seymour et al, supra). Thus the present invention may be used to reduce health risks to such patients from actual or potential periodontal infections.

Instead or in addition, the formulation of the invention may be for use in the treatment of dental caries. It may be for use against mutans streptococci, for example strains of S. mutans. It may be for use against a condition within the oral cavity which is caused, exacerbated or transmitted by (in particular either caused or transmitted by) such bacteria.

It is well known that mutans streptococci (MS) such as Streptococcus mutans and related bacteria (eg, Streptococcus sobrinus) are implicated as causative agents of dental caries (Loesch W J, 1986 Microbiological Reviews 50(4): 353-380; Islam B, Khan S N, Khan, 2007 Medical Science Monitor 13(11):196-203). This is particularly the case in children and other individuals that have a high sugar diet.

MS, especially S. mutans, are acid-tolerant and are highly cariogenic. They induce the formation of dental caries by the production of short chain carboxylic acids that demineralise and damage both tooth enamel and dentine. This effect is enhanced by close association between MS and the tooth surface in the form of a biofilm. The attachment of MS to the tooth surface is facilitated by their ability to produce extracellular polysaccharides such as glucans as a result of sugar metabolism. The attachment of Streptococcus mutans is further enhanced by the production of a specific adhesion protein (often referred to as PAc, antigen I/II, PI, and Spa PI), that is involved in both its primary attachment to the tooth and in providing further bacterial attachment sites. These factors contribute to the formation of a multi-species bacterial biofilm on the tooth surface. The biofilm is formed firstly by deposition of an acquired enamel pellicle comprising salivary and bacterial components, then adherence and co-adherence of multiple bacterial species of the oral cavity (including Actinomyces naeslundii) and finally the proliferation of these organisms. The biofilm provides a niche environment in which the cariogenic MS can thrive. It is notable that co-colonisation of the tooth surface by S. mutans and S. sobrinus leads to increased cariogenicity (Okada M, Soda Y, Hayashi F, 2005 Journal of Medical Microbiology 54: 661-665).

The present invention may be used to inhibit the growth of MS in the oral cavity and thereby reduce the cariogenic effects of these organisms. Furthermore the invention may be used to inhibit other bacterial components of the biofilm (for example Actinomyces naeslundii) adhering to the tooth surface, thus reducing biofilm integrity and its ability to provide a niche environment for cariogenic MS.

It has further been found that MS may be linked to (ie, may in case cause, increase susceptibility to and/or exacerbate) other more serious, often more systemic conditions. For example, MS have been linked with bacteraemia and its sequlae including but not limited to acute and subacute endocarditis (Verghagen D W M, Vedder A C, Speelman P, van der Meer, 2006 Journal of Antimicrobial Chemotherapy 57: 819-824). The present invention may accordingly be used, indirectly, to treat any such condition as well as to treat infections within the oral cavity and associated dental caries.

In an embodiment, the invented formulation may be for use in the treatment of an ocular infection. It may for example be used in the treatment of conjunctivitis due to Corynebacterium spp or in particular S. aureus, or in the treatment (in particular the prevention) of endophthalmitis due to Propionibacterium spp.

In an embodiment, the formulation may be for use in the treatment of an infection within the ear.

Infections of the eye and ear are commonly caused by Gram-positive bacteria (Kowalski R P, Dhaliwal D K, 2005 Expert Rev Anti Infect Ther. 3(1):131-9; de Miguel Martinez I, Ramos Macias A, Masgoret Palau E, 2007 Acta Otorrinolaringol Esp. 58(9):408-12). They are often caused by staphylococci, streptococci and occasionally by cutaneous propionibacteria. According to the fifth aspect of the invention, the invented formulation may be for use in the treatment of infections of the eye or ear since such combinations of compounds have been shown to be active against the following Gram-positive bacteria that are commonly identified as causative agents: Staphylococcus aureus (including MRSA, EMRSA, VISA and GISA), coagulase-negative staphylococci including S. auricularis, streptococci such as Streptococcus pyogenes and propionibacteria such as Propionibacterium acnes and Propionibacterium granulosum.

Infections of the eye or ear may also be caused by Gram-negative bacteria. In order to extend the activity of the invented formulation against such infections, it may contain, or be used in combination with, another antimicrobial agent, in particular a topical or systemic agent which is active against Gram-negative bacteria.

In an embodiment, the invented formulation may be for use in the treatment of an infection associated with an indwelling surgical device or implant, for example a catheter. Bacterial infections can frequently arise through the use of such devices (Wenzel R P, 2007 CID 45 (Suppl 1): S85-S88). This is particularly the case when Staphylococcus aureus, coagulase-negative staphylococci, streptococci, cutaneous propionibacteria (eg, in the case of artificial hip joints) and other bacteria adhere to the device and form a focus of infection and/or biofilm. Bacteria can detach from the initial infectious site and may be linked to (ie, may in cases cause, increase susceptibility to and/or exacerbate) other, systemic conditions such as bacteraemia and its sequlae, including for example acute and subacute endocarditis. Thus the formulation may be for use in the treatment of such infections and/or associated conditions.

Combinations of DASs or their derivatives with carbanilide antibacterial agents may thus be used in the treatment of infections associated with indwelling surgical devices, since such combinations have been shown to be active against S. aureus (including MRSA, EMRSA, VISA and GISA), coagulase-negative staphylococci (including but not restricted to S. auricularis, S. capitis, S. cohnii, S. epidermidis, S. haemolyticus, S. hominis, S. saprophyticus, S. simulans, S. warneri and S. xylosus), members of the genus Enterococcus (including E. faecalis), members of the genus Streptococcus (including. S. pyogenes), propionibacteria such as Propionibacterium acnes and Propionibacterium granulosum and members of the genus Corynebacterium (including C. mucifaciens).

Infections associated with catheters and other indwelling surgical devices may also be polymicrobial infections caused by both Gram-positive and Gram-negative bacteria. In such cases the invented formulation may be used in combination with another antimicrobial agent, in particular a topical or systemic agent which is active against Gram-negative bacteria.

In an embodiment, the invented formulation may be for use in the treatment of an infection of a wound (in particular a deep-seated wound), burn or ulcer. Deep-seated wounds, burns and ulcers are often infected by either Gram-positive bacteria, Gram-negative bacteria or mixed populations containing both types of bacteria (Hedrick T L, Smith P W, Gazoni L M et al, 2007, Current Problems in Surgery 44(10):635-75; Meara S O, Cullum,N, Majid M et al, 2000 Health Technology Assessment 4(21); Church D, Elsayed S, Reid O, 2006 Clinical Microbiology Reviews 19(2): 403-434; Anderson C A, Roukis T S, 2007 Surgical Clinics of North America 87:1149-1177). The invented combinations of compounds have been found capable of activity against a wide range of Gram-positive bacteria involved in such infections, and against certain anaerobic Gram-negative bacteria which can also be involved in these infections.

The invented formulation may be for use in the treatment of infected deep-seated wounds, burns and ulcers since such combinations of compounds have been shown to be active against the following Gram-positive bacteria that are commonly identified as the infective agents: Staphylococcus aureus (including MRSA, EMRSA, VISA and GISA), coagulase-negative staphylococci (including but not restricted to S. auricularis, S. capitis, S. cohnii, S. epidermidis, S. haemolyticus, S. hominis, S. saprophyticus, S. simulans, S. warneri and S. xylosus), members of the genus Streptococcus (eg, S. pyogenes), members of the genus Enterococcus (including E. faecalis), members of the genus Bacillus (eg, B. cereus), members of the genus Clostridium, propionibacteria such as Propionibacterium acnes and Propionibacterium granulosum, and members of the genus Corynebacterium (eg, C. mucifaciens). Surprisingly the invented formulation can also possess activity against the Gram-negative anaerobe Porphyromonas gingivalis, which may also be involved in infections of deep-seated wounds, burns and ulcers.

In an embodiment, the invented formulation is for use in the treatment of an infection within the throat. Throat infections can be caused by streptococci such as Streptococcus pyogenes or for instance by staphylococci such as Staphylococcus aureus (including MRSA, EMRSA, VISA and GISA). They may therefore be treated using the invented formulation since such combinations of compounds have been shown capable of activity against these causative organisms. A combination of a DAS or derivative and a carbanilide antibacterial agent may for example be incorporated into a mouthwash or other oral health care product (including a lozenge, pastille or other type of throat “sweet”), for the purpose of preventing, or reducing the risk of, a throat infection.

In an embodiment, the invented formulation may be for use in the treatment, in particular the prophylaxis, of an opportunistic infection. Immuno-compromised individuals who are otherwise susceptible to infection, for example due to HIV infection or other underlying diseases, malnutrition or the administration of immunosuppressive drugs, can be predisposed to opportunistic bacterial infections. Such infections can be caused by a wide range of Gram-positive and Gram-negative bacteria. The invented combinations have been found capable of activity against a wide range of Gram-positive bacteria involved in such infections and against certain anaerobic Gram-negative bacteria also involved in these infections. Thus they may be for use in the treatment of opportunistic infections since such combinations have been shown to be active against the Gram-positive bacteria which are commonly identified as the infective agents, for example: Staphylococcus aureus (including MRSA, EMRSA, VISA and GISA), coagulase-negative staphylococci (including S. auricularis, S. capitis, S. cohnii, S. epidermidis, S. haemolyticus, S. hominis, S. saprophyticus, S. simulans, S. warneri and S. xylosus), members of the genus Streptococcus (including S. pyogenes, S. mutans, S. sobrinus and S. pneumoniae), members of the genus Enterococcus (including E. faecalis), members of the genus Bacillus (eg, B. cereus), members of the genus Clostridium (eg, C. difficile or C. sporogenes), cutaneous propionibacteria such as Propionibacterium acnes and Propionibacterium granulosum, members of the genus Corynebacterium (including C. mucifaciens) and members of the family Actinomycetes (eg, Actinomyces naeslundii). For this purpose the invented formulation may for instance be applied to a dressing, surgical instrument, implant, catheter or the like to reduce the risk of bacterial infection during or after use of the item.

It has surprisingly also been found that combinations of DASs or their derivatives with carbanilide antibacterial agents can possess activity against the Gram-negative anaerobe Porphyromonas gingivalis, which also may be involved in opportunistic infections.

In an embodiment, the invented formulation may be for use in preventing the transmission of a food-borne bacterial pathogen, for example an infection caused by S. aureus, B. cereus, E. faecalis or Listeria monocytogenes. Moreover, as discussed below, in accordance with the invention the formulation may be used to control bacterial growth in order to inhibit, prevent or reduce food spoilage. Such combinations of compounds have-been shown to be active, or by inference from experimental data are believed to be active, against the following Gram-positive bacteria that are commonly identified as food spoilage agents and food borne pathogens: Staphylococcus aureus, (Mcluding MRSA, EMRSA, VISA and GISA), members of the genus Streptococcus, members of the genus Enterococcus (including E. faecalis and Enterococcus faecium), members of the genus Bacillus (eg, B. cereus), members of the genus Clostridium (eg, C. sporogenes), members of the genus Lactobacillus and Listeria monocytogenes (sole species).

In an embodiment, the invented formulation may be for use in the disinfection of skin or other tissue surfaces. Moreover, as described below, it may be used for the disinfection of non-living areas and surfaces. It may in particular be used to counter micro-organisms of the type referred to above. For example, it may be used to disinfect against the following Gram-positive bacteria: Staphylococcus aureus, members of the genus Enterococcus (including E. faecalis and E. faecium), members of the genus Bacillus (eg, B. cereus), members of the genus Clostridium (eg, C. sporogenes) and Listeria monocytogenes.

Surprisingly, the invented combinations of compounds have also been found capable of activity against the Gram-negative anaerobe Porphyromonas gingivalis, and may therefore be used to disinfect against this organism.

In an embodiment, the invented formulation may be for use in the treatment of a bacterial condition affecting an epithelial or mucosal surface such as in the nares, scalp, vagina, eyes, ears or oral cavity.

In an embodiment, the formulation is for use in the treatment of a condition which is caused, transmitted and/or exacerbated by (in particular either caused or transmitted by) a Gram-positive bacterium (whether aerobic or anaerobic), and/or by a bacterium selected from the group consisting of Gram-positive bacteria, P. gingivalis and Prevotella nigrescens, or in cases by a bacterium selected from the group consisting of Gram-positive bacteria and P. gingivalis.

In cases the formulation may be for use in the treatment of a condition which is caused, transmitted and/or exacerbated by (in particular either caused or transmitted by) an aerobic Gram-negative bacterium.

In an embodiment, the formulation is for use in the treatment of a condition selected from skin and skin structure conditions, in particular acne or superficial skin infections due to staphylococci and/or streptococci; body odour; and bacterial conditions within the oral cavity, such as periodontal diseases.

In an embodiment, the formulation is for use against one or more bacteria selected from staphylococci (in particular S. aureus and in cases also coagulase-negative staphylococci such as S. auricularis, S. capitis, S. cohnii, S. epidermidis, S. haemolyticus, S. hominis, S. saprophyticus, S. simulans, S. warneri and S. xylosus), members of the genus Streptococcus (in particular S. pyogenes), members of the genus Enterococcus (in particular E. faecalis), cutaneous propionibacteria (in particular P. acnes) and members of the genus Corynebacterium. In an embodiment, the formulation is for use in the treatment of a condition selected from acne, body odour and conditions affecting the oral cavity (in particular periodontal diseases). In an embodiment, the formulation is for use in the treatment of either acne or a condition affecting the oral cavity.

In certain cases, the invented formulation is not for use in the treatment of a polymicrobial or mixed infection, including in cases an infection which involves a Gram-negative bacterium and/or an infection which involves a fungus. In certain cases, it is not for use against a Gram-negative bacterium. In certain cases, it is not for use against a staphylococcal bacterium, in particular S. aureus.

According to the fifth aspect of the invention, the formulation of DAS or derivative and carbanilide antibacterial agent may be prepared in situ, at or immediately before the point of administration. This aspect of the invention thus pertains to any use of a DAS or pharmaceutically acceptable derivative thereof and a carbanilide antibacterial agent in the treatment of a bacterial condition, in particular acne, the two compounds being administered either simultaneously or sequentially.

According to a sixth aspect, the invention provides the use of a DAS or pharmaceutically acceptable derivative thereof and a carbanilide antibacterial agent, in the manufacture of a medicament (typically a formulation) for the treatment of a condition which is caused by, transmitted by and/or exacerbated by (in particular either caused or transmitted by) bacterial activity. The condition may be selected from those listed above in connection with the first to the fifth aspects of the invention. It may be a skin or skin structure condition, in particular acne. It may be body odour. It may be a bacterial infection within the oral cavity, in particular a periodontal disease. It may be a condition which is caused by, transmitted by and/or exacerbated by Gram positive cocci such as staphylococci. The DAS or derivative and the carbanilide will typically be used as an antibacterial combination in the manufacture of the medicament.

The invention further provides, according to a seventh aspect, the use together of a DAS or derivative thereof and a carbanilide antibacterial agent, as a combined antibacterial agent, or as a combined anti-acne agent, in the manufacture of an antibacterial formulation.

An eighth aspect provides a method for controlling the growth of a bacterium, in particular a propionibacterium, the method comprising applying, to an area or surface which is infected or suspected to be infected or capable of becoming infected with the bacterium, a combination of a DAS or a derivative thereof and a carbanilide antibacterial agent. Again the two compounds may be applied simultaneously or sequentially. They are suitably applied in a formulation of the type described above, preferably topically. They may in particular be applied to an area or surface which is infected with the relevant bacterium.

In this context, “controlling the growth” of a bacterium embraces inhibiting or preventing its growth, whether completely or partially, as well as killing either completely or partially a culture of the bacterium. It also embraces reducing the risk of subsequent growth of the bacterium in or on the area or surface being treated. It may embrace reducing the risk of transmission of the bacterium from the area or surface being treated to another area or surface and/or living body. The method of the invention may thus be used to treat an existing occurrence of the bacterium or to prevent a potential subsequent occurrence. Controlling the growth of a bacterium may also embrace the disruption and/or suppression of biofilm formation by the bacierium, as described above.

Again the area or surface to which the DAS or derivative and the carbanilide antibacterial agent are applied will typically be a surface such as human or animal tissue, in particular the skin or a tissue surface within the oral cavity, typically of a living human or animal. In this case the invented combination may be applied for therapeutic purposes or for non-therapeutic (eg, purely cosmetic) purposes. Thus the method of the eighth aspect of the invention encompasses a method of treatment of a human or animal patient suffering from or at risk of suffering from a condition which is caused by, transmitted by and/or exacerbated by (in particular either caused or transmitted by) bacterial, in particular propionibacterial, activity, the method involving administering to the patient a therapeutically (which term includes prophylactically) effective amount of an antibacterial formulation containing (a) a DAS or a pharmaceutically acceptable derivative thereof and (b) a carbanilide antibacterial agent. Again the bacterial condition may be any of those referred to above in connection with the first to the seventh aspects of the invention. The formulation is suitably administered in an antibacterially effective amount.

The method of the eighth aspect of the invention preferably involves applying a formulation according to the first aspect.

A ninth aspect of the invention provides the use of a DAS or derivative thereof in an antibacterial or anti-acne formulation, in combination with a carbanilide antibacterial agent, for the purpose of increasing the antibacterial and/or anti-acne activity of the formulation and/or of reducing the amount of the carbanilide in the formulation without undue loss of antibacterial or anti-acne activity.

An increase in antibacterial or anti-acne activity may be as compared to that of the carbanilide antibacterial agent alone, at the same concentration as used when combined with the DAS or derivative. Ideally the increase is as compared to the sum of the activities of the DAS or derivative and the carbanilide individually, again at the same respective concentrations as used when the two are combined.

A reduction in the amount of the carbanilide in the formulation may be as compared to the amount which would otherwise have been used in the formulation in order to achieve a desired level of activity, in particular in order to have acceptable efficacy in the context of its intended use. The reduction may be manifested by reduced side effects which would otherwise have been observed during use of the formulation, for example local irritation and/or undesirable systemic absorption of the carbanilide. According to the invention, the DAS or derivative may therefore be used for the dual purposes of reducing an undesired property of a formulation containing a carbanilide antibacterial agent, without undue loss of antibacterial or anti-acne activity.

Preferably the DAS or derivative is used without any reduction in antibacterial or anti-acne activity compared to the level exhibited by the formulation prior to addition of the DAS or derivative. More preferably it is used to give an increase in antibacterial or anti-acne activity. It may however be used to reduce the amount of the carbanilide present, and/or its associated side effects, whilst maintaining the antibacterial or anti-acne activity of the resultant formulation at a level, albeit lower than that which it would otherwise have exhibited, which is still acceptable in the context of its intended use.

A tenth aspect of the invention provides the use of a carbanilide antibacterial agent in an antibacterial or anti-acne formulation, in combination with a DAS or derivative thereof, for the purpose of increasing the antibacterial and/or anti-acne activity of the formulation and/or of reducing the amount of the DAS or derivative in the formulation without undue loss of antibacterial or anti-acne activity.

An increase in antibacterial or anti-acne activity may be as compared to that of the DAS or derivative alone, at the same concentration as used when combined with the carbanilide. Ideally the increase is as compared to the sum of the activities of the DAS or derivative and the carbanilide individually, again at the same respective concentrations as used when the two are combined.

A reduction in the amount of the DAS or derivative in the formulation may be as compared to the amount which would otherwise have been used in the formulation in order to achieve a desired level of-activity, in particular in order to have acceptable efficacy in the context of its intended use. The reduction may be manifested by reduced side effects which would otherwise have been observed during use of the formulation, for example local irritation and/or undesirable systemic absorption of the DAS or derivative. According to the invention, the carbanilide antibacterial agent may therefore be used for the dual purposes of reducing an undesired property of a formulation containing a DAS or a derivative thereof, without undue loss of antibacterial or anti-acne activity.

Preferably the carbanilide is used without any reduction in antibacterial or anti-acne activity compared to the level exhibited by the formulation prior to addition of the carbanilide. More preferably it is used to give an increase in antibacterial or anti-acne activity. It may however be used to reduce the amount of the DAS or derivative present, and/or its associated side effects, whilst maintaining the antibacterial or anti-acne activity of the resultant formulation at a level, albeit lower than that which it would otherwise have exhibited, which is still acceptable in the context of its intended use.

Further aspects of the invention

According to an eleventh aspect of the invention, there is provided an antibacterial or anti-acne formulation containing a dialkyl sulphosuccinate (DAS) or derivative thereof, wherein the concentration of the DAS or derivative is 3.5% w/w or greater. The formulation is preferably suitable and/or adapted for topical use.

The DAS or derivative may be of the type described above in connection with the first to the tenth aspects of the invention. Its two alkyl groups are suitably C₁ to C₁₂ alkyl groups, more suitably C₂ to C₁₀, such as C₆ to C₁₀ and in particular C₈. Thus the DAS is suitably a dioctyl sulphosuccinate (DOS) as described above. It may be used in the form of a salt such as an alkali metal or alkaline earth metal or ammonium salt, more particularly a sodium salt.

The concentration of the DAS or derivative in the formulation is suitably from 3.5 or 4 to 10% w/w or from 3.5 or 4 to 7% w/w, for example from 5 to 7% w/w. To our knowledge, these compounds have not previously been formulated at such high concentrations, in particular for topical use.

As discussed above, dialkyl sulphosuccinates are known as anionic surfactants and as wetting, solubilising, dispersing and emulsifying agents. Dioctyl sodium sulphosuccinate (DOS) in particular is known for use as a stabiliser and/or surfactant in formulations containing other active substances, including in anti-acne formulations. U.S. Pat. No. 4,497,794 for example, describes the use of DOS in a topical anti-acne composition, as a stabilising agent and surfactant, along with a synergistic mixture of an organic peroxide and erythromycin. The DOS concentration in the composition is said'to be from about 0.1 to 6% w/w, but the overall teachings of the document indicate a clear preference for lower concentrations; all its examples use 1% w/w or less. GB-2 054 375 discloses the use of DOS as a stabiliser and surfactant in an aqueous alcoholic peroxide-containing anti-acne gel, at a concentration from about 0.1 to 3% w/w, preferably from 0.1 to 1% w/w. US-2003/0044432 also describes an anti-acne formulation containing an oxidising agent such as benzoyl peroxide and an antibiotic such as erythromycin. This formulation can contain DOS as a surfactant, at a concentration of from 0.05 to 1% w/w; the surfactant is said to function as a processing aid and stabiliser.

In WO-86/07258 dialkyl sulphosuccinates, in particular dioctyl sodium sulphosuccinate, are also proposed for use in the treatment of bacterial infections of humans and animals. The document describes topical formulations for use as skin cleansers and as lotions for the treatment of acne. The dioctyl sodium sulphosuccinate is used however at relatively low concentrations, in the examples at 0.2 w/w. Potentially undesirable side effects are cited as a disincentive to using higher concentrations.

Moreover dioctyl sodium sulphosuccinate is also poorly water soluble which can limit its concentration in aqueous-based formulations. As a surfactant, the substance is typically used at a concentration of 1% w/w or lower. In skin formulations it is regarded by the FDA as an inactive ingredient when used at concentrations of 2 or 3% w/w.

It would therefore be desirable—in particular for the treatment of acne and related skin conditions—to be able to administer a dialkyl sulphosuccinate in a more optimised formulation, which contains a higher concentration of the active ingredient than has previously been possible, which has adequate stability even at that higher concentration and yet which is capable of releasing an effective quantity of the active, at the point of use. The eleventh aspect of invention seeks to address this need.

A twelfth aspect of the invention provides an antibacterial or anti-acne formulation which contains:

(i) a dialkyl sulphosuccinate (DAS) or derivative thereof;

(ii) a polyoxyalkylene-based solubilising agent;

(iii) an organic solvent, in particular an alcohol;

(iv) a thickening agent; and

(v) water.

It has surprisingly been found that this particular combination of ingredients can yield a stable formulation even at higher concentrations of the DAS active than have previously been thought feasible, the formulation being suitable for topical application in particular to the skin. The formulation may be particularly suitable for use in the treatment of skin and skin structure conditions such as acne. It may be suitable for use in the treatment of a staphylococcal infection. It may however be used for any of the purposes (both pharmaceutical and non-pharmaceutical) described above in connection with the first to the tenth aspects of the invention.

Preferred features of a formulation according to either the eleventh or the twelfth aspect of the invention may be as described above in connection with formulations according to the first aspect of the invention. This applies in particular to features such as the natures and concentrations of the constituents (i) to (v), its physical form (including its viscosity), its suitability for topical use, and any additional materials which it contains. It also applies to the nature and properties of the DAS or derivative.

The formulation may in particular be suitable and/or adapted and/or intended for topical administration to living tissue, in particular to the skin, to tissue surfaces within the nares or ears, and/or to tissue surfaces within the oral cavity.

In a formulation according to the twelfth aspect of the invention, the DAS or derivative is preferably present at a concentration of greater than 1-% w/w, more preferably 1.5 or 2 or 3 or 3.5 or 4% w/w or greater. It may be present at a concentration of up to 15%, w/w, or up to 10% w/w. Its concentration may for example be from 2 or 3.5 to 8% w/w, or from 3 or 3.5 to 7% w/w, or from 4 to 6% w/w, such as about 5% w/w.

According to a preferred embodiment, the invention therefore provides an antibacterial or anti-acne formulation which contains:

(i) from 2 to 8% w/w of a dialkyl sulphosuccinate (DAS) or derivative thereof;

(ii) from 10 to 30% w/w of a polyoxyalkylene-based solubilising agent;

(iii) from 10 to 30% w/w of an organic solvent, in particular an alcohol;

(iv) from 0.5 to 5% w/w of a thickening agent; and

(v) the balance water.

DAS-containing formulations of the type provided by the twelfth aspect of the invention are by no means, in the context of their intended use, straightforward combinations of standard excipients. It has been found, for example, that of the many commonly available solubilising agents, only a small number can be successfully used to formulate DASs into stable topical gels, in particular at the relatively high DAS concentrations preferred in the invention. The more conventional poloxamer solubilisers such as those in the Tween™ series, for example, have been found not to be suitable for use in the formulations of the invention. The use of solubilising agents such as Solutol® HS 15, which is more conventionally used in aqueous parenteral formulations rather than topical ones, is also far from intuitive in the context of a topical gel formulation and at the relatively high concentrations found to be preferred in a formulation according to the invention.

Particularly surprising is the fact that in the absence of the DAS, the remaining ingredients of the formulation appear not always to form a particularly stable mixture. There therefore appears to be a benefit, in terms of the stability of the overall formulations, due to the use of the invented combination of ingredients, which effect could not have been predicted from the prior art.

It may be preferred for the DAS or derivative to be the only active agent in a formulation according to either the eleventh or the twelfth aspect of the invention, or at least to be the only antibacterially active agent and/or the only anti-acne active agent.

It may be preferred for the formulation not to include benzyl alcohol. Benzyl alcohol is commonly used as a preservative in topical pharmaceutical formulations, but in some cases has been found to be detrimental to the stability of a DAS formulation according to the present invention.

In cases it may be preferred for the formulation not to contain a peroxide, in particular an organic peroxide, more particularly a diacyl peroxide such as benzoyl peroxide.

In cases it may be preferred for the formulation not to contain an antibiotic, in particular an antibiotic of the lincomycin family (most particularly clindamycin) as disclosed in U.S. Pat. No. 5,767,098 and/or erythromycin or a derivative thereof as disclosed in U.S. Pat. No. 4,497,794.

In cases the DAS or derivative may be used in the absence of a retinoid.

In accordance with the eleventh and twelfth aspects of the invention, the DAS or derivative is used as an active agent (ie, active as an antibacterial active against a skin or skin structure condition, in particular acne (which includes against a symptom and/or a cause of acne) and/or against one or micro-organisms associated with acne). It is suitably not used purely or even primarily as a stabiliser for another substance such as an active ingredient, or as a surface active agent, or as a wetting or solubilising or dispersing or emulsifying agent, or as a processing aid.

In particular, the DAS or derivative may be for use in treating acne and/or acne lesions. It may be for use in the treatment of a bacterial condition affecting the oral cavity, in particular a periodontal disease or dental caries. It may be for use in the treatment of a staphylococcal infection. It may be for use in the treatment of body odour.

A thirteenth aspect of the invention provides a product containing a formulation according to the eleventh or the twelfth aspect.

According to a fourteenth aspect, the invention provides a formulation according to the eleventh or the twelfth aspect, for use in the treatment of a condition affecting the human or animal body, which condition is caused by, exacerbated by and/or transmitted by (in particular caused or transmitted by) bacterial activity. The condition may be of the type described above in connection with the first to the thirteenth aspects of the invention. In particular it may be a skin or skin structure condition, and/or a condition which is caused, exacerbated or transmitted by propionibacterial activity. It may be acne, in which case the formulation may be for use as an anti-acne agent. It may be a staphylococcal infection. It may be a condition affecting the oral cavity.

A fifteenth aspect of the invention provides the use of a formulation according to the eleventh or the twelfth aspect, in the manufacture of a medicament for use in the treatment of a condition which is caused by, exacerbated by and/or transmitted by (in particular caused or transmitted by) bacterial activity. Again the condition may be as described above in connection with any of the previous aspects of the invention.

A sixteenth aspect provides the use of a DAS, or a pharmaceutically acceptable derivative thereof, in the manufacture of a medicament for use in the treatment of a condition which is caused by, exacerbated by and/or transmitted by (in particular caused or transmitted by) bacterial activity, wherein the DAS or derivative is present in the medicament at a concentration, of 3.5% w/w or greater. Again the DAS or derivative is suitably formulated as described above in connection with the previous aspects of the invention. Ther bacgerial condition may be as described above in connection with the previous aspects; it may in particular be a skin or skin structure condition, more particularly acne.

According to a seventeenth aspect of the invention there is provided a method for controlling the growth of a bacterium in particular a propionibacterium, the method comprising applying to an area or surface which is infected or suspected to be infected or capable of becoming infected with the bacterium, a formulation according to the eleventh or the twelfth aspect of the invention.

An eighteenth aspect provides a method of treatment of a human or animal patient suffering from or a risk of suffering from a condition which is caused by, transmitted by and/or exacerbated by (in particular caused or transmitted by) bacterial, in particular propionibacterial, activity, the method involving administering to the patient a therapeutically (which term includes prophylactically) effective amount of a formulation according to the eleventh or the twelfth aspect of the invention. Again the bacterial condition may be any of those referred to above in connection with the previous aspects of the invention. The formulation is suitably administered in an antibacterially effective amount.

It has also surprisingly been found that a formulation of the type described above may be active as an anti-acne agent in its own right, even in the absence of either a carbanilide or a DAS active agent.

Thus, a nineteenth aspect of the present invention provides an anti-acne formulation which contains:

(i) a polyoxyalkylene-based solubilising agent;

(ii) an organic solvent, in particular an alcohol;

(iii) a thickening agent; and

(iv) water,

in the absence of both DASs and derivatives thereof and TCC.

This formulation may contain one or more antimicrobial and/or anti-acne agents, in particular antibacterial agents. In cases however it may be free of other active agents in particular antimicrobial (especially antibacterial) agents and anti-acne agents. In cases it may be free of carbanilide antibacterial agents.

The natures and concentrations of the constituents of this formulation, and its physical form and/or suitability for topical application, may be as described above in connection with any of the previous aspects of the invention.

A twentieth aspect of the invention provides a formulation according to the nineteenth aspect, for use as an anti-acne agent. The formulation may in particular be for use to treat non-inflamed acne lesions. It is preferably suitable for topical application. In an embodiment, the formulation is not for use as an antimicrobial, in particular an antibacterial, agent. In an embodiment it may however be used in combination with an antimicrobial, in particular an antibacterial, agent.

A twenty-first aspect of the invention provides the use of a formulation according to the nineteenth aspect, in the preparation of a medicament (typically a formulation) for use in the treatment of acne. Again, the medicament may in particular be for use in the treatment of non-inflamed acne lesions, and is preferably suitable and/or adapted and/or intended for topical application.

A twenty-second aspect of the invention provides a product, for example a cosmetics or toiletry product, which incorporates a formulation according to the nineteenth aspect.

A twenty-third aspect provides a method of treatment of a patient who is suffering from or at risk of suffering from acne, in particular non-inflamed acne lesions, the method involving administering to the patient a therapeutically (which term includes prophylactically) effective amount of an anti-acne formulation according to the nineteenth aspect of the invention. The formulation is suitably administered topically.

Throughout the description and claims of this specification, the words “comprise” and “contain” and variations of the words, for example “comprising” and “comprises”, mean “including but not limited to”, and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith.

Moreover unless stated otherwise, any feature disclosed herein may be replaced by an alternative feature serving the same or a similar purpose.

The present invention will now be further described with reference to the following non-limiting examples.

### DETAILED DESCRIPTION

Experimental tests were conducted to determine the antibacterial activity of formulations according to the invention.

Test Micro-Organisms

One of the test micro-organisms used was Propionibacterium acnes NCTC 737. This is a propionibacterial strain and is the type strain of the genus; it is fully susceptible to antibiotics.

The propionbacteria are clinically significant due to their involvement in acne. This is a very common, complex and multi-factorial skin disease in which P. acnes and other Propionibacterium spp. (for example P. granulosum) play key roles. They are also opportunistic pathogens in compromised hosts. Thus, activity observed against these micro-organisms is expected to be a good predictor of activity against acne.

Other propionibacterial strains were also tested, as described in Example 3 and 12 below. These included certain antibiotic-resistant propionibacteria, such as the two P. acnes strains designated PRP-010 and PRP-039 which are resistant respectively to macrolides-lincosamides-streptogramins-ketolides (MLSK) and to macrolides lincosenides-streptogrammins (MLS) and tetracycline—in other words, PRP-010 is resistant to erythromycin and clindamycin, and PRP-039 to erythromycin, clindamycin and tetracycline.

In addition, certain strains of P. granulosum, another bacterium involved in acne, were also tested in Example 3 and 12.

The propionibacteria were cultured and maintained on Wilkins-Chalgren Anaerobe Medium (agar and broth) at pH 6.0; all cultures were incubated anaerobically at 37° C. for 72 hours.

Other microbial test species were used to demonstrate the suitability of formulations according to the invention for the treatment of conditions affecting the oral cavity. The first was Porphyromonas gingivalis, which is a black pigmented Gram-negative anaerobic bacterium belonging to the genus Porphyromonas. P. gingivalis is an oral pathogen typically associated with periodontal lesions, infections and adult periodontal disease. Gingivitis (inflammation of the gums that causes bleeding and exposes the base of the teeth) can be a precursor to periodontal disease by allowing P. gingivalis to infect the areas near the roots of the teeth and thus to cause tooth decay and infection.

Activity observed against this micro-organism is expected to be a reasonable qualitative predictor of antimicrobial activity against micro-organisms responsible for periodontal lesions and infections and periodontal disease. The principal test micro-organisms used were Por. gingivalis NCTC 11834 and Por. gingivalis 381 (the latter obtained from Professor H Kuramitsu, State University of Buffalo, Buffalo, N.Y., USA).

The P. gingivalis strains were cultured and maintained on Wilkins-Chalgren Anaerobe Medium (agar and broth) at pH 7.0; all cultures were incubated anaerobically at 37° C. for 5-7 days.

Also tested was Streptococcus mutans—this is a Gram-positive, microaerophilic bacterium associated principally with the human oral cavity. Clinically, S. mutans plays a significant role in dental caries and in infective endocarditis. The bacterium produces lactic acid as a by-product, of its normal metabolism and also produces an enzyme (dextransucrase) that can utilise sucrose to produce an extracellular based polysaccharide. This polysaccharide enables the bacteria to adhere to each other on the surface of a tooth to form plaque. It is the combination of the plaque and lactic acid that can result in tooth decay. More seriously, if the bacterium enters the bloodstream, for example after a tooth extraction, it can bind to the endocardium within the heart and if left untreated can prove fatal.

Activity observed against this micro-organism is thus expected to be a reasonable qualitative predictor of antibacterial activity against micro-organisms responsible for dental caries and infective endocarditis. Tests were performed with the strain Strep. mutans ATCC 25175.

The S. mutans was cultured and maintained on Wilkins-Chalgren Anaerobe Medium (agar and broth) at pH 7.0 supplemented with 1 g/L glucose; all cultures were incubated at 37° C. for 48 h in an atmosphere containing 5% CO₂.

Several Corynebacterium test species were also used in this study, namely C. mucifaciens ATCC 700355, C. striatum NCTC 764, C. xerosis NCTC 11861 and C. jeikeium NCTC 11915. These Gram-positive pathogens are closely related to organisms (aerobic diphtheroids of the genus Corynebacterium) that cause body odour. C. mucifaciens and C. striatum were cultured and maintained on Mueller-Hinton Medium (agar and broth) and were incubated aerobically at 37° C. for 24 hours. C. xerosis and C. jeikeium were cultured and maintained on Mueller-Hinton Medium containing 5% (v/v) lysed horse blood (agar and broth) and were incubated aerobically at 37° C. for 72 hours.

The following additional test organisms were also used:

| | | Culture conditions | | | |
|---|---|---|---|---|---|
| | Gram | Growth | Temp | | Time |
| Test organism | test | medium | (° C.) | Atmosphere | (h) |
| Bacillus cereus ATCC | + | MH | 30 | Aerobic | 24 |
| 11778 | | | | | |
| Bacteroides fragilis | − | WC pH7 | 37 | Anaerobic | 48 |
| ATCC 25285 | | | | | |
| Clostridium difficile | + | WC pH7 + | 37 | Anaerobic | 48 |
| ATCC 7000057 | | 5% lysed | | | |
| | | horse | | | |
| | | blood | | | |
| Enterococcus faecalis | + | MH | 37 | Aerobic | 24 |
| ATCC 29212 | | | | | |

[Abbreviations: American Type Culture Collection (ATCC), Mueller-Hinton (MH), Wilkins-Chalgren (WC). * denotes where agar medium was needed to maintain the organism 5% fresh horse blood was added to the medium.]

Of the above pathogens, B. cereus is associated with food poisoning; E. faecalis is a food borne pathogen and is implicated in burn and wound infections; and Clost. difficile may also be spread by contaminated food. The Bact. fragilis is a gut commensal but can act as an opportunistic pathogen eg, in peritoneal infections.

The following tests were carried out to assess antibacterial activity against the test organisms.

(a) Minimum Inhibitory Concentration (MIC) Assay

This is a standard international method for quantitatively assessing the antimicrobial activity of a compound in a liquid medium. The method used a sterile 96-well microtitre plate, capable of holding about 200 μl of liquid per well. The wells contained liquid culture medium and ranges of decreasing concentrations of the relevant test compound in doubling dilutions (eg, 1000, 500, 250, 125 . . . μg/ml, etc. down to 0.49 μg/ml). The culture media were as described above.

The wells were inoculated with a liquid suspension of freshly grown micro-organism and incubated under the conditions described above. After incubation, the microtitre plate was examined visually (with the aid of a light box) for cloudiness in each well, which would indicate microbial growth. The MIC value was recorded as the lowest concentration of test compound required to inhibit microbial growth, ie, the lowest concentration for which the liquid in the well remained clear.

The assays included both negative (culture medium with no micro-organisms) and positive (culture medium plus diluting solvent plus micro-organism) controls.

Since inhibition does not necessarily indicate killing of microbial cells, merely that growth as visible to the naked eye has been inhibited, it is desirable to conduct a further test (the MBC assay described below) to establish the concentration of the test compound needed to kill the test organism.

(b) Minimum Biocidal (Bactericidal) Concentration (MBC) Assay

This assay, normally carried out after an MIC assay, determines the rnininiurn concentration of a compound that is lethal to the micro-organism being tested.

Following an MIC assay, a 5 μl sample was withdrawn from the first microtitre well that showed positive growth and from all the subsequent wells that showed no growth. These samples were then individually sub-cultured on antibiotic-free agar medium, under the incubation conditions described above. Following incubation they were examined visually for microbial growth. The MBC was taken to be the lowest test compound concentration for which the incubated sample showed no growth.

The ratio of MIC to MBC should ideally be as close to 1 as possible. This facilitates selection of the lowest possible effective concentration of a test compound with a reduced risk of selecting a sub-lethal concentration which could promote resistance or allow the target microbial population to recover.

(c) Agar Dilution MIC Assay

This is a standard international method for quantitatively assessing the antimicrobial activity of a compound in a solid medium. The test compound was prepared to 40× the highest concentration required (eg, 10 mg/ml for a final concentration of 250 μg/ml) and a series of doubling dilutions were performed in a suitable solvent. A set amount of these antimicrobial stock solutions was then added to molten agar medium (ca. 55° C.), mixed thoroughly, poured into sterile Petri dishes and allowed to cool/set.

The culture medium was as described above.

A Multipoint™ Inoculator (AQS Manufacturing Ltd, UK) was used to inoculate the plates by spotting the inocula onto the surface of the agar, delivering approximately 1 to 2 μl per spot (yielding 10⁵ CFU(colony forming units) per spot).

The plate(s) were then incubated under the conditions described above, following which they were examined visually for signs of bacterial growth. The MIC value was ascertained when there was a marked reduction in, or total loss of, growth on the test plate at the lowest concentration as compared to that of the growth on the control plate.

The assays were conducted in duplicate and included a positive control (culture medium, diluting solvent and inoculum).

(d) Disc Diffusion Assay (DDA)

This is an internationally recognised standard method for qualitatively assessing the antimicrobial activity of a compound.

A sterile paper disc was impregnated with a sample of the test compound in a suitable solvent and 30 minutes allowed for the solvents to evaporate (where possible). The disc was then placed on an agar plate onto which the test micro-organism had been inoculated. The plate was then incubated under the conditions described above, following which it was examined visually for signs of microbial growth. If the test compound had antimicrobial activity, a circular zone of no growth would be obtained around the disc. The diameter of this zone of “inhibition” was measured using a ProtoCOL™ automated zone sizer (Synbiosis, Cambridge, UK). In general, a greater diameter and/or area of the zone of inhibition indicates a greater antimicrobial activity in the relevant test compound, although other factors such as test compound mobility through the agar gel may also influence the result.

(e) Synergy Disc Diffusion Assay (SDDA)

This is a variation on the DDA method, in which two compounds are tested together for their combined antimicrobal activity.

Two test a compounds A and B were placed on a single paper disc and the above described DDA procedure repeated. An increase in diameter of the zone of inhibition, compared to the greater of the zone diameters for the two compounds individually, was taken to indicate potential antimicrobial synergy. In practical terms, an increase of greater than 5 mm could be treated as significant.

(f) Supplemented Disc Diffusion Assays

Both the DDA and the SDDA test may be carried out using an agar gel supplemented with lipid and/or salt to simulate some of the major components present in human skin and to assess whether these substances might reduce the antimicrobial activity observed for the test compound. Performance under these conditions can provide a more reliable indication of activity on topical application. The supplements used in Examples 1, 11, 12 and 13 below were lipid (either Tween™ 80 at 1% v/v (Example 1) or triolein at 1% v/v (Examples 11, 12 and 13) and sodium chloride (100 mM).

(g) Biofilm Disruption Assay

This assay was designed to determine quantitatively the relative potency of a test compound to disrupt (effectively kill) biofilms formed by a test organism on hydroxyapatite (HA) discs.

Biofilms were formed on sterile dense ceramic HA discs 7 mm diameter x 1.8 mm thick (Clarkson Chromatography) by placing the discs in the appropriate liquid media (as described above) inoculated with the test organism (0.5×10⁸ cfu/ml). The HA discs were then incubated at 37° C. for ˜48 h in a 5% CO₂ atmosphere (for Strep. mutans ATCC 25175) or ˜96 h under anaerobic conditions (for Por. gingivalis 381). Following the incubation period the supernatant was removed from the wells containing the HA discs and the discs were washed×3 with fresh media to ensure all planktonic cells were removed. The HA discs were then transferred to sterile bijoux bottles containing 1 ml of phosphate buffered saline (PBS) plus test compound(s) at the required concentration. After a set time period (usually 2 and/or 5 min), the HA disc was transferred to another sterile bijoux containing 1 ml of fresh growth media. Adherent cells were then harvested by vigorous vortexing for 2 minutes. A sample was then taken, serially diluted 10-fold in fresh growth media and inoculated onto agar plates in triplicate. Untreated HA discs, and HA discs added to PBS containing only the solvents used to dissolve the test compound(s), acted as controls.

The plates were then incubated as described above and subsequently examined visually for growth. Colonies were counted at an appropriate serial dilution (5-50 individual colonies visible) with the aid of a colony counter. These measurements were then converted to numbers of colony forming units (cfu), using the formula: cfu/ml=number of colonies×serial dilution factor×100 (as only a 10 μl sample was taken).

These cfu values were then converted into log₁₀ values and plotted graphically against time of sample removal.

At each time point, samples were assessed in triplicate; the final cfu/ml value was an average (mean) of the three readings.

(h) Well Diffusion Assays

This is an alternative method for qualitatively assessing the antimicrobial activity of a compound.

A hole with a diameter of 8 mm was bored into the centre of an agar plate inoculated (swabbed) with the test-microorganism using a sterile “cork” borer (size No. 4). A volume (100 μl unless stated otherwise) of the test compound(s), either formulated or in a suitable solvent, was then placed into the bored hole. The plate was incubated under the conditions described above, following which it was examined visually for signs of microbial growth. If the test compound had antimicrobial activity, a circular zone of no growth would be obtained around the bored hole. The diameter of this zone of “inhibition” was measured using a ProtoCOL™ automated zone sizer (Synbiosis, Cambridge, UK). In general, a greater diameter and/or area of the zone of inhibition indicates a greater antimicrobial activity in the relevant test compound, although other factors such as test compound mobility through the agar gel, and the extent to which a compound is released from a formulation, may also influence the result.

### Example 1

### Activity against P. acnes (MIC, MBC & Disc Diffusion Assays)

The following experiments all used P. acnes NCTC 737 as the test organism.

MBC and DDA assays, as described above, were carried out using as the test compounds (a) the sodium salt of dioctyl sulphosuccinate (DOSS), dissolved in ethanol and (b) triclocarban (TCC) dissolved in DMSO. Both test compounds were sourced from Sigma Aldrich, UK.

Supplemented DDA assays were also carried out in the presence of salt and lipid.

Mixtures of the two test compounds were then subjected to SDDA assays as described above, including in the presence of salt and lipid. Increases in zone diameter (mm) were measured with respect to the TCC, which was the compound showing the larger zones of inhibition during the previous individual disc diffusion assays.

For the (S)DDA experiments, including the supplemented versions, 50 μg of the DOSS was loaded onto each disc, and 200 μg of the TCC. All the (S)DDA experiments were conducted in triplicate.

The MIC and MBC results are shown in Table 1 below and the (S)DDA results in Tables 2 (unsupplemented assays) and 3 (assays supplemented with salt and lipid). All results are collated from a number of experiments.

TABLE 1

| | MIC | MBC |
|---|---|---|
| Test compound | (μg/ml) | (μg/ml) |
| DOSS | 15.6 | 62.5 |
| TCC | 0.12 | 31.25 |

TABLE 2
(unsupplemented assays)

| | | SDDA | | SDDA |
|---|---|---|---|---|
| | | with | SDDA | area |
| | DDA | DOSS | increase | increase |
| Test compound | (mm) | (mm) | (mm) | (%) |
| DOSS | 11.81 | | | |
| | (±2.43) | | | |
| TCC | 18.76 | 29.95 | 11.19 | 154.9 |
| | (±0.72) | (±1.26) | | |

TABLE 3
(assays supplemented with salt & lipid)

| | | SDDA | | |
|---|---|---|---|---|
| | | with | SDDA | |
| | DDA | DOSS | increase | SDDA area |
| Test compound | (mm) | (mm) | (mm) | increase (%) |
| DOSS | 0.00 | | | |
| TCC | 0.00 | 10.57 | 10.57 | ∞ |
| | | (±0.00) | | |

These data show that both the DAS and the carbanilide are active on their own against P. acnes NCTC 737, although not, at the concentrations used, in supplemented disc diffusion assays.

Surprisingly, however, when the two compounds are combined the data indicate a synergistic antibacterial interaction between them, with a significant increase in zone diameter and area over those exhibited by either compound alone. This synergy is also observed under the supplemented conditions, providing an antibacterial effect where the individual test compounds alone did not.

### Example 2

### Activity against P. acnes (other DASs)

The general method of Example 1 was repeated using two further DASs, the calcium and potassium salts of dioctyl sulphosuccinate (DOS). Both were obtained from Badrivishal Chemicals & Pharmaceuticals, India. These were tested against P. acnes NCTC 737, both alone and in combination with TCC. The DOS salts were dissolved in ethanol and and the TCC in DMSO.

For the (S)DDA experiments, 200 μg of each test compound was loaded onto each disc.

The MIC and MBC results are shown in Table 4 below and the (S)DDA results in Table 5. All results are collated from a number of experiments.

TABLE 4

| | MIC | MBC |
|---|---|---|
| Test compound | (μg/ml) | (μg/ml) |
| DOS calcium salt | 15.6 | >31.25 |
| DOS potassium salt | 7.8 | 62.5 |

TABLE 5

| | | SDDA | SDDA | |
|---|---|---|---|---|
| | DDA | with TCC | increase | SDDA area |
| Test compound | (mm) | (mm) | (mm) | increase (%) |
| TCC | 15.69 | | | |
| | (±1.41) | | | |
| DOS calcium salt | 23.9 | 36.16 | 12.26 | 128.93 |
| | (±0.48) | (±0.54) | | |
| DOS potassium salt | 25.91 | 36.68 | 10.7 | 99.37 |
| | (±0.78) | (±0.36) | | |

The data in Tables 4 and 5 show that other DASs may be combined with a carbanilide antibacterial agent to provide a synergistic level of antibacterial activity against P. acnes NCTC 737. This indicates the potential of such combinations for use as anti-acne agents.

### Example 3

### Activity against other Propionibacteria

Using DOSS and TCC as the test compounds, the activity of each compound alone and in combination was determined against a number of other Propionibacterium spp strains, including some with known antibiotic resistance.

For the (S)DDA experiments, 50 μg of the DOSS and/or 200 μg of the TCC were loaded onto each disc. All the (S)DDA experiments were conducted in triplicate.

The results are shown in Table 6 below; the resistance phenotype for each of the test strains is also indicated.

TABLE 6

| | | | | SDDA | | SDDA |
|---|---|---|---|---|---|---|
| | | DDA | DDA | TCC + | SDDA | area |
| | Resistance | TCC | DOSS | DOSS | increase | increase |
| Test organism | phenotype | (mm) | (mm) | (mm) | (mm) | (%) |
| P. granulosum NCTC 11865 | None | 17.78 | 14.70 | 30.11 | 12.33 | 186.84 |
| | | (±3.77) | (±0.78) | (±0.78) | | |
| P. acnes PRP-002 | Tet/MLS | 34.22 | 31.76 | 37.00 | 2.77 | 16.87 |
| | | (±1.41) | (±2.22) | (±2.22) | | |
| P. acnes PRP-003 | Tet | 23.64 | 27.95 | 33.50 | 5.55 | 43.65 |
| | | (±1.08) | (±0.71) | (±0.18) | | |
| P. acnes PRP-004 | Tet | 15.42 | 26.62 | 35.76 | 9.15 | 80.53 |
| | | (±0.82) | (±1.46) | (±0.92) | | |
| P. granulosum PRP-005 | MLSK | 12.13 | 26.31 | 26.21 | −0.10 | −0.78 |
| | | (±0.18) | (±0.78) | (±0.82) | | |
| P. granulosum PRP-006 | MLS | 10.17 | 19.01 | 27.64 | 8.63 | 111.43 |
| | | (±0.92) | (±0.78) | (±0.71) | | |
| P. acnes PRP-007 | Clin | 13.87 | 21.38 | 38.44 | 17.06 | 223.31 |
| | | (±3.7) | (±3.04) | (±2.8) | | |
| P. acnes PRP-008 | Clin | 14.80 | 24.87 | 34.94 | 10.07 | 97.39 |
| | | (±0.53) | (±3.04) | (±1.28) | | |
| P. acnes PRP-010 | MLSK | 22.81 | 26.82 | 35.04 | 8.22 | 70.70 |
| | | (±0.53) | (±0.53) | (±0.36) | | |
| P. acnes PRP-017 | MLS | 18.81 | 28.98 | 39.36 | 10.38 | 84.46 |
| | | (±0.31) | (±0.82) | (±0.94) | | |
| P. granulosum PRP-019 | MLSK | 0.00 | 19.94 | 27.34 | 7.40 | 88.00 |
| | | (±0.0) | (±3.12) | (±1.52) | | |
| P. granulosum PRP-021 | MLS | 0.00 | 0.00 | 30.32 | 30.32 | ∞ |
| | | (±0.0) | (±0.0) | (±1.39) | | |
| P. acnes PRP-023 | MLSK | 21.27 | 26.41 | 39.36 | 12.95 | 122.09 |
| | | (±1.63) | (±3.04) | (±0.36) | | |
| P. acnes PRP-026 | MLS | 22.71 | 28.26 | 35.25 | 6.99 | 55.57 |
| | | (±0.47) | (±1.08) | (±0.36) | | |
| P. acnes PRP-039 | Tet/MLS | 18.91 | 23.02 | 38.02 | 15.00 | 172.84 |
| | | (±0.47) | (±0.64) | (±1.58) | | |
| P. granulosum PRP-043 | MLS | 8.84 | 8.94 | 30.52 | 21.58 | 1065.40 |
| | | (±0.47) | (±0.53) | (±0.0) | | |
| P. granulosum PRP-044 | MLS | 0.00 | 0.00 | 27.23 | 27.23 | ∞ |
| | | (±0.0) | (±0.0) | (±0.89) | | |
| P. acnes PRP-046 | None | 20.25 | 25.08 | 35.66 | 10.59 | 102.25 |
| | | (±0.64) | (±1.98) | (±1.08) | | |
| P. acnes PRP-053 | Tet/MLS | 21.58 | 17.78 | 36.38 | 14.80 | 184.16 |
| | | (±0.82) | (±2.62) | (±2.69) | | |
| P. granulosum PRP-055 | None | 17.16 | 10.07 | 37.10 | 19.94 | 367.28 |
| | | (±0.36) | (±0.36) | (±2.35) | | |
| P. acnes PRP-059 | MLS | 19.73 | 11.82 | 36.59 | 16.85 | 243.79 |
| | | (±0.31) | (±2.17) | (±0.78) | | |
| P. acnes PRP-068 | Ery | 22.30 | 25.59 | 38.13 | 12.54 | 122.00 |
| | | (±2.1) | (±3.43) | (±1.55) | | |
| P. acnes PRP-101 | Tet/MLS | 16.75 | 14.59 | 37.61 | 20.86 | 404.18 |
| | | (±0.47) | (±6.03) | (±0.53) | | |
| P. acnes PRP-102 | Tet/MLS | 14.59 | 22.81 | 39.15 | 16.34 | 194.54 |
| | | (±0.36) | (±5.61) | (±2.41) | | |

[Abbreviations: American Type Culture Collection (ATCC), National Collection of Type Cultures (NCTC), Propionibacterium Panel Number (PRP), Tetracycline (Tet), Erythromycin (Ery), Clindamycin (Clin), Macrolide-Lincosamide-Streptogramin (MLS), Macroliode-Lincosamide-Streptogramin-Ketolide (MLSK).]

When the DOSS and TCC are combined, the synergistic interaction initially observed with P. acnes NCTC 737 was similarly observed, with the exception P. granulosum PRP-005, against all of the propionibacteria tested. This indicates the utility, of the combination either to treat or to prevent infections associated with such bacteria, in particular acne. The results are likely to be of particular clinical value for the antibiotic resistant test strains.

### Example 4

### Activity against S. aureus

The activities of DOSS and TCC against S. aureus ATCC 29213 and EMRSA 15 were determined using (S)DDAs as described above. 200 μg of the DOSS and/or the TCC were loaded onto each disc. All the experiments were conducted in triplicate.

The results are shown in Table 7 below. All results are collated from a number of experiments.

TABLE 7

| | | | SDDA | | SDDA |
|---|---|---|---|---|---|
| | DDA | DDA | TCC + | SDDA | area |
| | TCC | DOSS | DOSS | increase | increase |
| Test organism | (mm) | (mm) | (mm) | (mm) | (%) |
| S. aureus ATCC 29213 | 12.84 | 12.22 | 20.08 | 7.25 | 144.8 |
| | (±0.18) | (±1.0) | (±0.65) | | |
| EMRSA 15 | 12.42 | 12.42 | 19.15 | 6.73 | 137.7 |
| | (±0.0) | (±0.82) | (±0.9) | | |

These data indicate that a DAS can be combined with a carbanilide antibacterial agent to give a synergistic level of activity against both sensitive and antibiotic resistant strains of S. aureus. The data indicate the likely utility of the combination in the prevention and/or treatment of staphylococcal infections such as MRSA.

### Example 5

### Activity Bacteria against Bacteria Associated with Oral Health Conditions

The activities of DOSS and TCC were determined, using (S)DDAs, against Por. gingivalis NCTS 11834 and Strep. mutans ATCC 25175. In addition the calcium and potassium salts of dioctyl sulphosuccinate (DOS) were also tested against Por. gingivalis.

For the (S)DDA experiments; 200 μg of the DOS salts and/or the TCC were loaded onto each disc for Por. gingivalis experiments. For the Strep. mutans experiments, 200 μg of the TCC and 50 μg of DOSS were used on each disc. All the experiments were conducted in triplicate.

The (S)DDA results for Por. gingivalis and Strep. mutans are shown below in Tables 8 and 9 respectively. All results are collated from a number of experiments.

TABLE 8

| | | SDDA | SDDA | |
|---|---|---|---|---|
| | DDA | with TCC | increase | SDDA area |
| Test compound | (mm) | (mm) | (mm) | increase (%) |
| TCC | 23.73 | | | |
| | (±1.01) | | | |
| DOSS | 51.13 | 63.52 | 12.39 | 54.34 |
| | (±1.79) | (±1.73) | | |
| DOS calcium salt | 52.07 | 61.31 | 9.24 | 38.64 |
| | (±2.02) | (±2.99) | | |
| DOS potassium salt | 51.13 | 61.31 | 10.18 | 43.78 |
| | (±1.31) | (±1.62) | | |

TABLE 9

| | | SDDA | SDDA | |
|---|---|---|---|---|
| | DDA | with TCC | increase | SDDA area |
| Test compound | (mm) | (mm) | (mm) | increase (%) |
| TCC | 11.95 | | | |
| | (±0.78) | | | |
| DOSS | 24.1 | 28.78 | 4.68 | 42.61 |
| | (±1.57) | (±3.45) | | |

Tables 8 and 9 show that a DAS can be combined with a carbanilide antibacterial agent to give a synergistic level of activity against both Por. gingivalis NCTC 11834 and Strep. mutans ATCC 25175. This in turn indicates the likely utility of such combinations in treating bacterial infections within the oral cavity, in particular periodontal diseases and dental caries, and/or in reducing the build-up of plaque.

### Example 6

### Activity against Corynebacterium spp

The activities of DOSS and TCC were determined, using (S)DDAs, against the test organisms C. mucifaciens ATCC 700355, C. striatum NCTC 764, C. xerosis NCTC 11861 and C. jeikeium NCTC 11915. 200 μg of the DOSS and/or the TCC were loaded onto each disc. All the experiments were conducted in triplicate.

The results are shown in Table 10 below. All results are collated from a number of experiments.

TABLE 10

| | | | SDDA | | |
|---|---|---|---|---|---|
| | | | with | SDDA | SDDA area |
| | Test | DDA | TCC | increase | increase |
| Test strain | compound | (mm) | (mm) | (mm) | (%) |
| C. mucifaciens | TCC | 9.59 | | | |
| | | (±0.18) | | | |
| | DOSS | 12.92 | 21.05 | 8.13 | 165.45 |
| | | (±0.48) | (±0.18) | | |
| C. striatum | TCC | 14.08 | | | |
| | | (±0.36) | | | |
| | DOSS | 19.57 | 25.57 | 6.0 | 70.79 |
| | | (±0.0) | (±0.72) | | |
| C. xerosis | TCC | 11.65 | | | |
| | | (±0.36) | | | |
| | DOSS | 0.0 | 18.25 | 6.6 | 145.4 |
| | | (±0.0) | (±0.31) | | |
| C. jeikeium | TCC | 0.0 | | | |
| | | (±0.0) | | | |
| | DOSS | 0.0 | 13.81 | 13.81 | ∞ |
| | | (±0.0) | (±0.18) | | |

Table 10 shows that a DAS can be combined with a carbanilide antibacterial agent to give a synergistic level of activity against all the Corynebacterium species tested. This in turn indicates the likely utility of such combinations in treating bacterial infections which are associated with body odour, in particular in the axilla or feet. Such combinations may therefore be used to treat (which includes to prevent) body odour. They may suitably be applied topically, for example in the form of a deodorant or anti-perspirant, powder or other skin care formulation. They may be incorporated into or coated on a shoe insert such as an insole or sock.

### Example 7

### Activity against other Micro-Organisms

The activities of DOSS and TCC were determined, using (S)DDAs, against a panel of further test micro-organisms, namely B. cereus ATCC 11778, Bact. fragilis ATCC 25285, Clost. difficile ATCC 7000057 and E. faecalis ATCC 29212. 200 μg of the DOSS and/or the TCC were loaded onto each disc. All the experiments were conducted in triplicate.

The results are shown in Table 11 below. All results are collated from a number of experiments.

TABLE 11

| | | | SDDA | | SDDA |
|---|---|---|---|---|---|
| | DDA | DDA | TCC + | SDDA | area |
| | TCC | DOSS | DOSS | increase | increase |
| Test organism | (mm) | (mm) | (mm) | (mm) | (%) |
| B. cereus ATCC 11778 | 12.96 | 18.10 | 22.31 | 4.21 | 51.93 |
| | (±0.81) | (±0.18) | (±1.24) | | |
| Bact. fragilis ATCC | 14.71 | 19.74 | 33.11 | 13.37 | 181.34 |
| 25285 | (±0.18) | (±1.93) | (±0.64) | | |
| Clost. difficile ATCC | 0.0 | 0.0 | 14.54 | 14.54 | ∞ |
| 7000057 | (±0.0) | (±0.0) | (±1.24) | | |
| E. faecalis ATCC 29212 | 9.68 | 10.31 | 17.68 | 7.37 | 194.07 |
| | (±0.18) | (±0.36) | (±0.0) | | |

The Table 11 data show that a DAS can be combined with a carbanilide antibacterial agent to give a synergistic level of activity against clinically important Gram-positive (aerobic and anaerobic) and Gram-negative (anaerobic only, in these tests) bacteria. This in turn indicates the likely utility of such combinations in treating bacterial infections caused by these micro-organisms.

### Example 8

### Topical Anti-Acne Formulations

The results from Examples 1 to 3 show that the combination of a dialkyl sulphosuccinate and a carbanilide antibacterial agent such as TCC can be an effective antibacterial agent, in particular against the bacteria associated with acne, with a synergistic impact on the antibacterial activity of the combination compared to those of the individual compounds alone. This can be of use in preparing antibacterial formulations, in particular for topical application to the skin, for either prophylactic or therapeutic use in any context where such bacteria are thought to be involved as possible sources of infection. More specifically, it can be of use in preparing anti-acne formulations, again suitably for topical use.

Even in cases where a combination of a DAS or derivative thereof with a carbanilide antibacterial agent has an additive, as opposed to synergistic, antibacterial activity compared to that of the individual compounds, this can be of considerable benefit when preparing formulations for topical use: One of the compounds may be used to replace a proportion of the other, thus lowering any side effects and/or other undesirable properties of the combination without undue loss of antibacterial activity.

A topical formulation for use in treating acne may for example be prepared by combining a DAS such as a DOS, or a pharmaceutically acceptable salt thereof such as dioctyl sodium sulphosuccinate, with a carbanilide antibacterial agent such as TCC or a pharmaceutically acceptable salt thereof, in a suitable fluid vehicle and optionally together with conventional additive. Such vehicles and additives may be for instance as found in Williams' “Transdermal and Topical Drug Delivery” Pharmaceutical Press, 2003 and other similar reference books, and/or in Rolland A et al, “Site-specific drug delivery to pilosebaceous structures using polymeric microspheres”, Pharm. Res. 1993; 10: 1738-44; Mordon S et al, “Site-specific methylene blue delivery to pilosebaceous structures using highly porous nylon microspheres: an experimental evaluation”, Lasers Surg. Med. 2003; 33: 119-25; and Alvarez-Roman Ret al, “Skin penetration and distribution of polymeric nanoparticles”, J. Controlled Release 2004; 99: 53-62.

The formulation may be prepared and administered using known techniques. It may for example take the form of a cream, lotion or in particular a gel.

The concentrations of the two active agents may be in the ranges described above, and will be determined based on the intended use of the formulation, its intended mode of administration and the activities of the particular chosen active agents.

By way of example, gel formulations for use in the topical treatment of acne may be prepared using the following ingredients (all figures quoted are percentages by weight).

TABLE 12

| | Example A | Example B | Example C | Example D | Example E | Example F | Example G |
|---|---|---|---|---|---|---|---|
| [symbol] ctyl sodium | 5 | 5 | 5 | 5 | 2.5 | 2.5 | 5 |
| [symbol] hosuccinate | | | | | | | |
| [symbol] | 0.2 | 0.2 | 0.2 | 0.2 | 0.2 | 0.2 | 0.5 |
| [symbol] tol ® HS15 | 20 | 20 | | | 20 | | |
| [symbol] cerox ™ 767 | | | 20 | 15 | | 20 | 20 |
| [symbol] nol | 20 | 20 | 20 | 20 | 15 | 15 | 20 |
| [symbol] roxyethyl cellulose | | 1 | 1 | 1 | 1 | 1 | |
| [symbol] ium carboxymethyl | 1 | | | | | | 1 |
| [symbol] lose | | | | | | | |
| [symbol] er | Balance | Balance | Balance | Balance | Balance | Balance | Balance |

[symbol]

indicates data missing or illegible when filed

These formulations may be prepared using standard chemical formulation techniques. For example, the following general preparation method may be used.
1. Add the required amount of thickening agent to water with continuous (for example propeller) mixing.
2. In a separate vessel, mix the DAS or derivative and the organic solvent, suitably in dehydrated form, and stir for instance using a magnetic stirrer until the DAS or derivative is completely dissolved.
3. In a third vessel, mix the solubilising agent and the carbanilide. Heat if necessary, with continuous stirring, until the solubilising agent has melted and the carbanilide is completely dissolved. For example, the Solutol® HS 15 used in the examples above may be heated to 50° C.±5° C. in order to liquefy it. Then cool the mixture to an appropriate temperature for mixing with the other ingredients, suitably maintaining it in liquid form (for example, for Solutol® HS 15, to between 30 and 35° C.).
4. Mix the cooled carbanilide solution of step 3 with the DAS solution formed in step 2, for instance using a magnetic stirrer.
5. Add the mixture formed in step 4 to the water/thickening agent mixture formed in step 1. Mix well, for example using a propeller.

In all of the above formulations, methanol or isopropanol, in particular methanol, may be used instead of ethanol, as may a mixture of two or more such alcohols or indeed any other suitable organic solvent or mixture thereof. An alternative thickening (preferably gelling) agent, suitably another cellulosic material or a carbomer, may be used in place of the hydroxyethyl cellulose or sodium carboxymethyl cellulose. An alternative polyoxyalkylene based solubilising agent may be used in place of the Solutol® HS 15 or Glycerox™ 767. An alternative DAS or derivative may be used in place of the dioctyl sodium sulphosuccinate. An alternative carbanilide antibacterial agent may be used in place of the TCC.

Further ingredients may be incorporated into the formulations, for example as described above.

The formulations may be administered topically to acne-affected skin.

### Example 9

### Stability Tests

The stability of the Example B formulation was evaluated by storing samples, in sealed glass scintillation vials, for six weeks at three different temperatures in ambient humidity. At the beginning and end of the storage period, the physical appearance and pH of each sample were recorded, and the DAS and TCC contents were determined by HPLC.

The results are shown in Table 13 below.

TABLE 13

| | T = 0 weeks | | | T = 6 weeks | | |
|---|---|---|---|---|---|---|
| | 5° C. | 25° C. | 40° C. | 5° C. | 25° C. | 40° C. |
| [symbol] S assay | 100 | 100 | 100 | 101 | 100.8 | 99.8 |
| [symbol] ult (%) | | | | | | |
| [symbol] C assay | 100 | 100 | 100 | 93.8 | 93.7 | 93.0 |
| [symbol] ult (%) | | | | | | |
| [symbol] pearance | Clear, | Clear, | Clear, | Clear, | Clear, | Clear, |
| | colourless | colourless | colourless | colourless | colourless gel | colourless |
| | gel | gel | gel | gel | | gel |
| | 6.45 | 6.45 | 6.45 | 6.23 | 6 | 5.43 |

[symbol]

indicates data missing or illegible when filed

These data indicate the long term stability of a DAS/carbanilide gel formulation according to the invention, despite the inclusion of a much higher than usual concentration of the DAS and in addition the carbanilide co-active.

### Example 10

### Topical Anti-Staphylococcal Formulations

The results from Example 4 show that combinations of DASs and carbanilides can be effective anti-staphylococci agents. This can be of use in preparing antibacterial formulations, in particular for topical application to the skin, for prophylactic or therapeutic use in any context where such bacteria are thought to be involved as possible sources of infection, for example in the nares or ears or indeed on the hands.

A formulation for use against staphylococci such as S. aureus may be prepared by combining a DAS such as DOSS, or a (preferably pharmaceutically acceptable) derivative thereof, with a carbanilide antibacterial agent such as TCC, in a similar manner to that described for the anti-acne formulations of Example 8. The ingredients may in this case be formulated as a spray, for instance for application to work surfaces or surgical instruments; as a cleansing gel or lotion for instance for hand washing; as a nasal spray for application to the anterior nares; as ear or eye drops; or in many other appropriate forms. Such a formulation may in particular be used prophylactically, eg, to reduce the risk of outbreaks of MRSA or similar infections.

### Example 11

### Activity against P. acnes—DOSS Alone

The following experiments all used P. acnes NCTC 737 as the test organism.

Liquid-phase MIC assays, and MBC and DDA assays, as described above, were carried out using as the test compound the sodium salt of dioctyl sulphosuccinate (DOSS, ex Sigma Aldrich, UK), dissolved in ethanol.

The DDA assays were then repeated in the presence of lipid and salt.

For the DDA experiments, 200 μg of the test compound, dissolved in ethanol, was loaded onto each disc. These assays were carried out at a pH of 6.0. All the DDA experiments were conducted as single replicates.

The results are shown in Table 14 below; all are collated from a number of experiments.

TABLE 14

| | |
|---|---|
| MIC (μg/ml) | 15.6 |
| MBC (μg/ml) | 62.5 |
| MIC/MBC ratio | 0.25 |
| DDA (mm) | 47.63 |
| DDA + salt (mm) | 45.14 |
| DDA + lipid (mm) | 47.32 |

It can be seen from Table 14 that the dioctyl sulphosuccinate sodium salt is highly active as an antibacterial agent against P. acnes NCTC 737. This indicates its likely activity as an anti-acne agent, the propionibacteria being implicated in acne.

The antibacterial activity is maintained in the presence of both salt and lipid, indicating the suitability of the test compound for the topical treatment of acne.

### Example 12

### Activity against other Propionibacteria

Example 11 was repeated (except for the MBC assays) against a number of other Propionibacterium spp strains, including some with known antibiotic resistance. Again DOSS was used as the test compound. MICs were determined by agar dilution and DDA assays were carried out as described above, for each of the strains.

The results are shown in Table 15 below; all are collated from a number of experiments. The table also indicates the resistance phenotype for each of the test strains.

TABLE 15

| | | | | DDA + | DDA + |
|---|---|---|---|---|---|
| | Resistance | MIC | DDA | salt | lipid |
| Test organism | phenotype | (μg/ml) | (mm) | (mm) | (mm) |
| P. granulosum | None | 31.25 | 39.84 | 37.98 | 38.60 |
| NCTC 11865 | | | | | |
| P. acnes PRP-002 | Tet/MLS | 15.6 | 49.49 | 51.36 | 48.25 |
| P. acnes PRP-003 | Tet | 15.6 | 42.96 | 46.69 | 43.58 |
| P. acnes PRP-004 | Tet | 15.6 | 48.56 | 43.27 | 46.07 |
| P. granulosum PRP-005 | MLSK | 15.6 | 42.96 | 46.38 | 40.47 |
| P. granulosum PRP-006 | MLS | 31.25 | 40.47 | 41.09 | 40.16 |
| P. acnes PRP-007 | Clin | 15.6 | 47.32 | 42.96 | 44.82 |
| P. acnes PRP-008 | Clin | 15.6 | 48.25 | 48.56 | 43.58 |
| P. acnes PRP-010 | MLSK | 15.6 | 46.69 | 46.07 | 39.84 |
| P. acnes PRP-017 | MLS | 15.6 | 47.94 | 47.63 | 44.82 |
| P. granulosum PRP-019 | MLSK | 15.6 | 41.71 | 42.02 | 42.65 |
| P. granulosum PRP-021 | MLS | 31.25 | 39.53 | 38.60 | 42.02 |
| P. acnes PRP-023 | MLSK | 15.6 | 49.49 | 47.32 | 46.38 |
| P. acnes PRP-026 | MLS | 15.6 | 49.81 | 48.25 | 43.89 |
| P. acnes PRP-039 | Tet/MLS | 15.6 | 47.63 | 46.38 | 47.32 |
| P. granulosum PRP-043 | MLS | 31.25 | 40.16 | 35.80 | 35.18 |
| P. granulosum PRP-044 | MLS | 31.25 | 38.29 | 37.67 | 35.49 |
| P. acnes PRP-046 | None | 15.6 | 44.51 | 47.00 | 41.09 |
| P. acnes PRP-053 | Tet/MLS | 15.6 | 49.49 | 44.82 | 42.02 |
| P. granulosum PRP-055 | None | 31.25 | 40.16 | 38.29 | 36.42 |
| P. acnes PRP-059 | MLS | 15.6 | 43.58 | 44.20 | 44.82 |
| P. acnes PRP-068 | Ery | 15.6 | 48.87 | 46.69 | 44.51 |
| P. acnes PRP-101 | Tet/MLS | 15.6 | 41.40 | 45.14 | 40.47 |
| P. acnes PRP-102 | Tet/MLS | 31.25 | 44.82 | 41.71 | 37.98 |

[Abbreviations: American Type Culture Collection (ATCC), National Collection of Type Cultures (NCTC), Propionibacterium Panel Number (PRP), Tetracycline (Tet), Erythromycin (Ery), Clindamycin (Clin), Macrolide-Lincosamide-Streptogramin (MLS), Macroliode-Lincosamide-Streptogramin-Ketolide (MLSK).]

Table 15 shows that the dioctyl sulphosuccinate sodium salt is active against a range of different Propionibacterium spp strains. This indicates its utility either to treat or to prevent infections associated with such bacteria, in particular acne. The results are likely to be of particular clinical value for the antibiotic resistant test strains.

### Example 13

### Activity against P. acnes—other DASs

Example 11 was repeated, but using as the test compounds the calcium and potassium salts of dioctyl sulphosuccinate (both ex Sigma Aldrich, UK). The results are shown in Table 16 below.

TABLE 16

| | | | | | DDA + | DDA + |
|---|---|---|---|---|---|---|
| | MIC | MBC | MIC/MBC | DDA | salt | lipid |
| Test compound | (μg/ml) | (μg/ml) | ratio | (mm) | (mm) | (mm) |
| DOS calcium | 15.6 | >31.25 | <0.5 | 44.71 | 43.16 | 49.36 |
| salt | | | | | | |
| DOS potassium | 7.8 | 62.5 | 0.125 | 46.57 | 46.26 | 48.12 |
| salt | | | | | | |

Table 16 shows that other DASs are also active against P. acnes NCTC 737, and therefore also of potential utility as anti-acne agents.

### Example 14

### Activity against other Micro-Organisms

Example 11 was repeated, using sodium (DOSS), calcium and potassium dioctyl sulphosuccinates as the test compounds, against a number of other test organisms.

For the DDA experiments, 200 μg of the test compound was loaded onto each disc. The DOS salts were all dissolved in ethanol. The DDA experiments were conducted in triplicate, with the exception of calcium and potassium DOS against S. mutans which were single replicates.

The results are shown in Table 17 below; all are collated from a number of experiments.

TABLE 17

| | | | | MIC/ | |
|---|---|---|---|---|---|
| | Test | MIC | MBC | MBC | DDA |
| Test organism | Compound | (μg/ml) | (μg/ml) | ratio | (mm) |
| Por. gingivalis NCTC | DOSS | <0.49 | <0.49 | n/a | 51.13 |
| 11834 | | | | | (±1.79) |
| Strep. mutans ATCC | | 15.6 | 15.6 | 1 | 40.25 |
| 25175 | | | | | (±3.4) |
| C. striatum NCTC 764 | | 62.5 | 62.5 | 1 | 19.57 |
| | | | | | (±0.0) |
| Por. gingivalis NCTC | DOS | 3.9 | 7.8 | 0.5 | 52.07 |
| 11834 | calcium salt | | | | (±2.02) |
| Strep. mutans ATCC | | 15.6 | 31.25 | 0.5 | 32.13 |
| 25175 | | | | | |
| Por. gingivalis NCTC | DOS | 3.9 | 7.8 | 0.5 | 51.13 |
| 11834 | potassium | | | | (±1.31) |
| Strep. mutans ATCC | salt | 15.6 | 31.25 | 0.5 | 35.28 |
| 25175 | | | | | |

These results show that dialkyl sulphosuccinates may be used as antibacterial agents against Por. gingivalis, Strep. mutans and C. striatum. This also indicates their likely utility in treating bacterial infections within the oral cavity, in particular periodontal diseases and dental caries, and/or in reducing the build-up of plaque, and/or in treating bacterial infections which are associated with body odour.

### Example 15

### Activity against Por. gingivalis & Strep. mutans (Biofilm Disruption Assays)

The following experiments were conducted using Por. gingivalis 381 and Strep. mutans ATCC 25175 as the test organisms.

Biofilm disruption assays, as described above, were carried out using DOSS as the test compound and chlorhexidine (CHX) as a positive control. For these experiments, the DOSS was tested at 0.5% w/v against Por. gingivalis and 1% w/v against Strep. mutans. The ahlorhexidine was tested at a concentration of 0.2% w/v against both micro-organisms. The DOSS was dissolved in ethanol and the chlorhexidine in distilled water. All the experiments were conducted in triplicate.

The results for Por. gingivalis are shown in Table 18 and those for Strep. mutans in Table 19.

TABLE 18

| | Viable cell counts (log₁₀ cfu/ml) | | |
|---|---|---|---|
| Treatment | 0 min | 2 min | 5 min |
| 0.5% (w/v) DOSS | 7.53 | 5.08 | 3.34 |
| | (±0.21) | (±0.89) | (±1.47) |
| 0.2% (w/v) CHX | 7.53 | 6.50 | 6.76 |
| | (±0.21) | (±0.40) | (±0.32) |
| Control | 7.53 | 7.53 | 7.28 |
| | (±0.21) | (±0.25) | (±0.07) |

TABLE 19

| | Viable cell counts (log₁₀ cfu/ml) | | |
|---|---|---|---|
| Treatment | 0 min | 2 min | 5 min |
| 1.0% (w/v) DOSS | 6.56 | 2.87 | 3.36 |
| | (±0.2) | (±0.54) | (±1.61) |
| 0.2% (w/v) CHX | 6.56 | 6.12 | 5.99 |
| | (±0.2) | (±0.33) | (±0.23) |
| Control | 6.56 | 6.25 | 6.39 |
| | (±0.2) | (±0.41) | (±0.31) |

The tabulated data demonstrate the activity of the DOSS against sessile bacteria (ie, bacteria present in a biofilm) against both test organisms They also show its activity to be significantly greater than that of the more conventionally used oral health care active chlorhexidine.

These results indicate the likely suitability of the test compound for the treatment of either Por. gingivalis or Strep. mutans infections in vivo. It may therefore be used to disrupt and/or suppress biofilm formation by the relevant bacteria, and/or to reduce, kill or at least inhibit bacterial colonies established within biofilms, for instance in the oral cavity.

### Example 16

### Topical Anti-Acne Formulations

The results from Examples 11 to 13 show that a DAS can be an effective antibacterial agent, in particular against the bacteria associated with acne. This can be of use in preparing antibacterial formulations, in particular for topical application to the skin, for either prophylactic or therapeutic use in any context where such bacteria are thought to be involved as possible sources of infection. More specifically, it can be of use in preparing anti-acne formulations, again suitably for topical use.

Gel formulations for use in the topical treatment of acne may be prepared using the following ingredients (all figures quoted are percentages by weight).

TABLE 20

| | Exam- | Exam- | Exam- | Exam- | Exam- |
|---|---|---|---|---|---|
| | ple | ple | ple | ple | ple |
| | I | II | III | IV | V |
| Sodium dioctyl | 5 | 5 | 5 | 5 | 5 |
| sulphosuccinate | | | | | |
| Solutol ™ HS 15 | 20 | 20 | | 20 | |
| Glycerox ™ 767 | | | 20 | | 20 |
| Dehydrated ethanol | 20 | 20 | 20 | 15 | 15 |
| Hydroxyethyl cellulose | | 1 | 1 | 1 | 1 |
| Sodium carboxymethyl | 1 | | | | |
| cellulose | | | | | |
| Water | Balance | Balance | Balance | Balance | Balance |

These formulations may be prepared using standard chemical formulation techniques. For example, the following general preparation method may be used.
1. Add the required amount of thickening agent to water with continuous (for example propeller) mixing.
2. In a separate vessel, mix the DAS or derivative and the organic solvent, suitably in dehydrated form, and stir for instance using a magnetic stirrer until the DAS or derivative is completely dissolved.
3. In a third vessel, heat the solubilising agent if necessary, with continuous stirring, until it has completely melted. For example, the Solutol® HS 15 used

TABLE 21

| | T = 0 | | | T = 6 | | |
|---|---|---|---|---|---|---|
| | weeks | | | weeks | | |
| | 5° C. | 25° C. | 40° C. | 5° C. | 25° C. | 40° C. |
| DAS assay | 100 | 100 | 100 | 101 | 100.8 | 99.8 |
| result (%) | | | | | | |
| Appearance | Clear, | Clear, | Clear, | Clear, | Clear, | Clear, |
| | colourless | colour- | colour- | colour- | colour- | colour- |
| | gel | less | less | less | less | less |
| | | gel | gel | gel | gel | gel |
| pH | 6.45 | 6.45 | 6.45 | 6.23 | 6 | 5.43 |

These data indicate the stability of a gel formulation according to the invention, despite the inclusion of a much higher than usual concentration of the dialkyl sulphosuccinate salt.

### Example 18

### Activity of Formulated DOSS against P. acnes

The activities of (a) the formulation of Example 16 (II), containing 5% w/w of dioctyl sodium sulphosuccinate, 20% w/w Solutol™ HS 15, 20% w/w ethanol, 1% w/w hydroxyethyl cellulose and the balance water; (b) a “placebo” as above but without DOSS; and (c) DOSS dissolved solely in ethanol (5% w/v) were determined against P. acnes NCTC 737 using a well diffusion assay as previously described.

The results are shown in Table 22 below. Values represent the mean of triplicate replicates.

TABLE 22

| | DDA |
|---|---|
| Test formulation | (mm) |
| (a) DOSS (5% w/w) in | 62.97 |
| formulation | (±3.38) |
| (b) Placebo | 0.0 |
| | (±0.0) |
| (c) DOSS (5% w/v) in | 56.31 |
| ethanol | (±2.52) |

in the examples above may be heated to 50° C.±5° C. in order to liquefy it. Then cool the solubilising agent to an appropriate temperature for mixing with the other ingredients, suitably maintaining it in liquid form (for example, for Solutol® HS 15, to between 30 and 35° C.).
4. Mix the cooled solubilising agent with the DAS solution formed in step 2, for instance using a magnetic stirrer.
5. Add the mixture formed in step 4 to the water/thickening agent mixture formed in step 1. Mix well, for example using a propeller.

In all of the above formulations, methanol or isopropanol, in particular methanol, may be used instead of ethanol, as may a mixture of two or more such alcohols or indeed any other suitable organic solvent or mixture thereof. An alternative thickening (preferably gelling) agent, suitably another cellulosic material or a carbomer, may be used in place of the hydroxyethyl cellulose or sodium carboxymethyl cellulose. An alternative polyoxyalkylene-based solubilising agent may be used in place of the Solutol® HS 15 or ^{Glycerox™} 767. An alternative DAS or derivative thereof may be used in place of the dioctyl sodium sulphosuccinate.

Further ingredients may be incorporated into the formulations, for example as described above. The formulations may be administered topically to acne-affected skin.

### Example 17

### Stability Tests

The stability of the Example II formulation was evaluated by storing samples, in sealed glass scintillation vials, for six weeks at three different temperatures in ambient humidity. At the beginning and end of the storage period, the physical appearance and pH of each sample were recorded, and the DAS content was determined by HPLC.

The results are shown in Table 21 below.

It can be seen that the DOSS is active in its own right against P. acnes NCTC 737, when dissolved at 5% w/w in ethanol. However, the same concentration of DOSS but contained within the invented formulation (a) demonstrates a markedly increased level of activity. This indicates the likely utility of the invented formulation in the treatment of acne.

### Example 19

### Topical Oral Health Care Formulations

Examples 5, 14 and 15 show that DASs, both alone and in combination with carbanilide antibacterial agents, can be active against bacteria associated with oral health problems, in particular Por. gingivalis and Strep. mutans. This indicates the utility of such actives or combinations in treating infections caused by the relevant test organisms, in particular infections within the oral cavity such as for example plaque formation, gingivitis, periodontitis and dental caries. Such actives and combinations may also be used for general oral health care, for example for the creation and/or maintenance of fresh-smelling breath. They may also be used to treat more systemic conditions associated with Strep. mutans or Por. gingivalis infections, for example infective endocarditis or cardiovascular diseases.

A topical formulation for use in this way, for instance against S. mutans and/or P. gingivalis, may be prepared by formulating a DAS or a pharmaceutically acceptable derivative thereof, for example a dioctyl sulphosuccinate, either alone or with a carbanilide antibacterial agent such as TCC, in a suitable fluid vehicle and optionally together with conventional additives, as described above.

The formulation may be prepared and administered using known techniques. For topical application it may for example take the form of a paste, cream, gel, lozenge, buccal patch, spray, mouthwash or dentifrice, or it may be carried in or on a dental fibre or tape. It may contain additives which target the active ingredient(s) to a particular site, for example the gums or teeth or the sub-gingival regions, and/or which otherwise control the release of the active(s) at the relevant site.

### Example 20

### Topical Anti-BO Formulations

Examples 6 and 14 show that DASs, both alone and in combination with carbanilide antibacterial agents, can be active against bacteria associated with body odour. This can be of use in preparing antibacterial formulations, in particular for topical application to the skin, for either prophylactic or therapeutic use in any context where such bacteria are thought to be involved as possible sources of infection. More specifically, it can be use in preparing formulations for use against body odour in particular in the axilla and/or feet, again suitably for topical use.

Even in cases where a combination of a DAS or derivative thereof with a carbanilide antibacterial agent has an additive, as opposed to synergistic, antibacterial activity compared to that of the individual compounds, this can be of considerable benefit when preparing formulations for topical use. One of the compounds may be used to replace a proportion of the other, thus lowering any side effects and/or other undesirable properties of the combination without undue loss of antibacterial activity.

A topical formulation for use in the treatment of body odour may be prepared by formulating a DAS or a pharmaceutically acceptable derivative thereof, for example a dioctyl sulphosuccinate, with a carbanilide antibacterial agent such as TCC, in a suitable fluid vehicle and optionally together with conventional additives, as described above.

The formulation may be prepared and administered using known techniques. It may for example take the form of a roll-on, spray or “stick” anti-perspirant or deodorant formulation, or of a dusting powder such as a talcum powder, or of a gel or cream or ointment. It may contain an anti-perspirant and/or deodorant agent, and/or a fragrance. It may be coated on or incorporated into a sock or shoe, or a shoe insole.

### Example 21

### further DAS/Carbanilide Formulations

Solutions of DOSS and TCC (both ex Sigma Aldrich) were prepared using various concentrations of the two actives. All formulations contained, in addition to the DOSS and TCC, (i) 20% w/w Solutol™ HS 15 (ex BASF) and (ii) 20% w/w anhydrous ethyl alcohol (Sigma Aldrich), and were made up to 100% with purified water (Sigma Aldrich).

These solutions were diluted 1:2 using a placebo solution containing 20% w/w Solutol™ HS 15, 20% w/w anhydrous ethyl alcohol and the balance purified water (ie, no DAS or carbanilide actives present). They were then tested in triplicate for their antibacterial potency against P. acnes NCTC 737 via disc diffusion assays (DDAs) and synergy disc diffusion assays (SDDAs), as previously described 10 μl of each test solution was loaded onto each paper disc;

The results are shown in Table 23 below. A “+” in the interaction column indicates that antibacterial synergy was observed.

TABLE 23

| | | | | (S)DDA |
|---|---|---|---|---|
| | % DOSS | (S)DDA | | increase |
| % TCC (w/w) | (w/w) | (mm) | Interaction | (mm) |
| 0 | 0 | 0.00 | | |
| 0 | 2.5 | 0.00 | | |
| 0 | 5 | 31.88 | | |
| 0 | 7.5 | 40.52 | | |
| 0.1 | 0 | 0.00 | | |
| 0.2 | 0 | 0.00 | | |
| 0.3 | 0 | 30.10 | | |
| 0.1 | 2.5 | 0.00 | 0 | — |
| 0.1 | 5 | 35.21 | + | 3.33 |
| 0.1 | 7.5 | 43.96 | + | 3.44 |
| 0.2 | 2.5 | 29.79 | + | 29.79 |
| 0.2 | 5 | 35.94 | + | 4.06 |
| 0.2 | 7.5 | 43.65 | + | 3.13 |
| 0.3 | 2.5 | 34.69 | + | 4.59 |
| 0.3 | 5 | 39.48 | + | 7.6 |
| 0.3 | 7.5 | 46.46 | + | 5.94 |

These data show that in this particular formulation, both the DOSS and the TCC are active on their own against P. acnes NCTC 737, although not at concentrations of 2.5% w/w and below for DOSS and 0.2% w/w and below for TCC.

Surprisingly, however, when the two compounds are combined the data indicate a synergistic antibacterial interaction between them, with significant increases in zone diameter over those exhibited by either compound alone. This effect is observed over a range of TCC:DOSS concentration ratios. A particularly strong potentiation is seen for the combination of 0.2% w/w TCC with 2.5 w/w DOSS.

### Example 22

### Activity of DOSS (+TCC) in Gel Formulations against P. acnes

Gel formulations according to the invention were prepared using either DOSS or TCC alone (both ex Sigma Aldrich) or combinations of the two. The gels contained, in addition to the two active ingredients, (i) 20% w/w Solutol™ HS 15 (BASF), (ii) 20% w/w anhydrous ethyl alcohol (Sigma Aldrich) and (iii) 1% hydroxyethyl cellulose (Sigma Aldrich), and were made up to 100% with purified water (Sigma Aldrich).

These gels were then diluted 1:2 using a placebo gel as in Example 18, and tested in triplicate for their antibacterial potency against P. acnes NCTC 737 via a well diffusion assay as previously described. The results are shown in Table 24 below.

TABLE 24

| | | | | (S)DDA |
|---|---|---|---|---|
| | | % DOSS | (S)DDA | increase |
| Gel | % TCC w/w | w/w | (mm) | (mm) |
| Placebo | 0 | 0 | 0 | |
| DOSS | 0 | 5 | 42.40 | |
| TCC | 0.2 | 0 | 0 | |
| DOSS + | 0.2 | 5 | 48.13 | 5.73 |
| TCC | | | | |

It can be seen that the DOSS is active in its own right against P. acnes NCTC 737, when present at 5 w/w in this particular formulation. The TCC however, at a concentration of 0.2% w/w, shows no activity against the bacterium using this assay. This is also the case for the placebo gel which contains neither TCC nor DOSS.

Surprisingly, however, when the two compounds are combined in the gel formulation, the data indicate a synergistic antibacterial interaction between them, with a significant increase in zone diameter over those exhibited by either compound alone.

This experiment also illustrates the ability of a gel formulation according to the invention to release a DAS or combined DAS/carbanilide active agent at the point of use.

### Example 23

### Trial Data

A trial was carried out on human patients to assess the anti-acne activity of formulations according to the invention. The four test formulations were:
A the formulation of Example 16(II), containing 5% w/w of dioctyl sodium sulphosuccinate, 20% w/w Solutol™ HS 15, 20% w/w ethanol, 1% w/w hydroxyethyl cellulose and the balance water.
B the formulation of Example 8(B), containing 5% w/w of dioctyl sodium sulphosuccinate, 0.2% w/w TCC, 20% w/w Solutol™ HS 15, 20% w/w ethanol, 1 w/w hydroxyethyl cellulose and the balance water.
C an excipient combination similar to that used in the Example 8(B) formulation, containing 20% w/w Solutol™ HS 15, 20% w/w ethanol, 1% w/w hydroxyethyl cellulose and the balance water (ie, no DAS or carbanilide active agents).
D as a positive control, a commercially available anti-acne product (L'Oreal™ Pure Zone Deep Control Moisturiser, purchased in the EU). This contained salicylic acid, which is known to be effective against non-inflamed acne lesions.

The trial was a randomised, controlled trial with four arms, and was performed double-blind for the test treatments and single-blind for the positive control. Its primary aim was to compare the efficacy of the four test formulations in subjects with acneic skin.

The trial was carried out in six centres throughout Germany; people of either sex with acne of at least grade 3 on the eight point scale of Allen & Smith [Allen B S, Smith J G, “Various parameters for grading acne vulgaris”, Arch Dermatol. 1982, 118: 23-25] were eligible to participate. Treatment (approximately 0.5 g) was applied by the participants once a day for eight weeks, to the entire face. No concomitant anti-acne medications were permitted and previous treatment was stopped at least four weeks before the baseline visit. There were between 27 and 31 patients in each of the four test groups, and each group was assigned one of the four test formulations.

Inflamed and non-inflamed lesions were counted at 8, 15, 29 and 57 days after treatment began. Formulation success rates were also assessed on each of these occasions using (a) a “physician's global assessment” and (b) a “global satisfaction score” allocated by the participants themselves. (a) was carried out by medical practitioners using the Allen & Smith scale referred to above, and took account of initial acne severity. (b) was assessed using a 6-point Likert-like scale where 1=extremely dissatisfied and 6=extremely satisfied. The reference for this scale is Rapp D A et al, “Anger and Acne: Implication for Quality of Life, Patient Satisfaction and Clinical Care”, Br J Dermatol 2004, 151: 183-89. Participants rated how well the relevant product worked, its ease of use and the time spent using it.

The results are shown in Table 25 below, for mean inflamed lesion counts (INFL), mean non-inflamed lesion counts (NINFL), mean total lesion counts (LES), mean physician's global assessment scores (PGA) and mean subjects' global satisfaction scores (SGSS). The figures quoted are percentage changes from the baseline (ie, the mean counts or scores at the baseline visit prior to starting treatment), apart from the SGSS values which represent the mean scores at each assessment visit.

TABLE 25

| Meas- | | | | | |
|---|---|---|---|---|---|
| ured | | Formulation | Formulation | Formulation | Formulation |
| property | Day | A | B | C | D (control) |
| INFL | 8 | −4.72 | −10.92 | −14.45 | −16.08 |
| | 15 | −1.09 | −20.74 | −13.17 | −18.02 |
| | 29 | −8.38 | −24.67 | −10.26 | −24.55 |
| | 57 | −23.4 | −32.64 | −13.64 | −15.54 |
| NINFL | 8 | −2.71 | −6.30 | −4.63 | −9.72 |
| | 15 | −14.10 | −17.40 | −13.74 | −5.15 |
| | 29 | −16.17 | −28.63 | −17.62 | +2.19 |
| | 57 | −22.86 | −24.18 | −25.82 | −8.86 |
| LES | 8 | −3.40 | −8.08 | −8.51 | −12.53 |
| | 15 | −9.91 | −18.69 | −13.52 | −10.80 |
| | 29 | −13.48 | −27.10 | −14.71 | −9.59 |
| | 57 | −23.04 | −27.44 | −21.01 | −11.67 |
| PGA | 8 | −2.56 | −10.75 | −9.56 | −14.05 |
| | 15 | −12.07 | −21.49 | −12.18 | −12.18 |
| | 29 | −19.83 | −25.62 | −17.39 | −17.39 |
| | 57 | −34.48 | −38.02 | −25.22 | −25.22 |
| SGSS | 8 | 63.0 | 60.0 | 37.9 | 70.0 |
| | 15 | 66.7 | 77.4 | 55.2 | 76.7 |
| | 29 | 77.8 | 74.2 | 62.1 | 63.3 |
| | 57 | 77.8 | 83.9 | 69.0 | 70.0 |

Table 25 shows the following:
that the DAS-containing test formulations according to the invention (formulations A and B) had achieved a greater reduction in inflamed lesion counts, by the end of the trial, than the positive control (formulation D). The combination of the DAS with the carbanilide appeared to be the most effective in this respect.
that all three of the formulations according to the invention had achieved a markedly greater reduction in non-inflamed lesion counts, by the end of the trial, than the positive control. In this context formulation C, which according to the nineteenth aspect of the invention contained neither DAS nor carbanilide, appeared to be the most effective.
that in terms of total lesion counts (the mean change in numbers of all lesions, whether inflamed or not, from the baseline), all three formulations according to the invention had performed as well as if not better than the positive control by the end of the eight week trial period.
that the physician's global assessment scores indicate a better overall performance by the two DAS-containing test formulations, A and B, than by the positive control, and a performance for the DAS-free formulation C which is at least comparable with that of the control.
that by the end of the trial, subject satisfaction scores were high for all the formulations according to the invention, in particular the DAS-containing formulations, and compared favourably with those for the positive control.

The end-of-trial changes versus the baseline were statistically significant for the two DAS containing formulations A and B, across all outcomes as well those for formulation C for all outcomes except the inflamed lesion count.

The four test formulations were ranked for efficacy across the different outcomes, with 1 representing the best in each case and 4 the worst. The results are summarised in Table 26 below.

TABLE 26

| Measured | Formulation | | | Formulation |
|---|---|---|---|---|
| property | A | Formulation B | Formulation C | D (control) |
| INFL | 2 | 1 | 4 | 3 |
| NINFL | 3 | 2 | 1 | 4 |
| LES | 2 | 1 | 3 | 4 |
| PGA | 2 | 1 | 3 | 4 |
| SGSS | 2 | 1 | 4 | 3 |

It can be seen that the two DAS-containing formulations ranked more favourably than the control across all of the measured outcomes, including both efficacy and patient satisfaction. Formulation B, containing both DAS and carbanilide, fared particularly well in this regard. Formulation C, according to the nineteenth aspect of the invention, ranked higher than the control in the context of non-inflamed lesion counts.

These data demonstrate the utility of formulations according to the invention in the topical treatment of acne. In particular, they show that a formulation according to the nineteenth aspect of the invention can be surprisingly effective in the treatment of non-inflamed acne lesions, despite the absence of. DAS or carbanilide antibacterial agents and despite its apparently low antibacterial activity (propionibacteria being associated with inflamed but not with non-inflamed acne lesions). In this respect the formulation according to the invention appears to perform at least as well as the commercially available salicylic-acid containing product.

It is of note that the trial participants reported no treatment-related local side effects for any of the four test treatments, showing that the formulations according to the invention were well tolerated.

77. An antibacterial or anti-acne formulation containing a dialkyl sulphosuccinate (DAS) or derivative thereof, wherein the concentration of the DAS or derivative is 3.5% w/w or greater.

78. A formulation according to claim 77, which additionally contains:
(i) a polyoxyalkylene-based solubilising agent;
(ii) an organic solvent;
(iii) a thickening agent; and
(iv) water.

79. A formulation according to claim 77, which is suitable for topical application to human skin.

80. A formulation according to claim 77, wherein the DAS or derivative is in the form of a metal salt.

81. A formulation according to claim 77, wherein the DAS is a dioctyl sulphosuccinate.

82. A formulation according to claim 77, which is in the form of a cream, paste, gel, lotion, foam, ointment, varnish or other viscous or semi-viscous fluid.

83. A formulation according to claim 78, wherein the solubilising agent is polyoxyethylene (polyethylene glycol, PEG)-based.

84. A formulation according to claim 78, wherein the solubilising agent comprises a polyoxyalkylene ether or ester.

85. A formulation according to claim 78, wherein the solubilising agent is selected from (a) polyalkoxylated esters, other than polyalkoxylated sorbitan esters and (b) polyalkoxylated alkyl ethers.

86. A formulation according to claim 78, wherein the organic solvent is an alcohol.

87. A product containing an antibacterial or anti-acne formulation according to claim 77.

88. A method of treatment of a human or animal patient suffering from or at risk of suffering from a condition which is caused by, transmitted by and/or exacerbated by bacterial activity, the method involving administering to the patient a therapeutically (which term includes prophylactically) effective amount of an antibacterial or anti-acne formulation according to claim 77.

89. A method according to claim 88, wherein the condition is acne and/or acne lesions.

90. An antibacterial or anti-acne formulation which contains:
(i) a dialkyl sulphosuccinate (DAS) or derivative thereof;
(ii) a polyoxyalkylene-based solubilising agent;
(iii) an organic solvent, in particular an alcohol;
(iv) a thickening agent; and
(v) water.

91. A method of treatment of a human or animal patient suffering from or at risk of suffering from a condition which is caused by, transmitted by and/or exacerbated by bacterial activity, the method involving administering to the patient a therapeutically (which term includes prophylactically) effective amount of a formulation according to claim 90.
